(19) United States
(12) Patent Application Publication
(10) Pub. No.: US 2025/0197479 A1
(43) Pub. Date: Jun. 19, 2025

(54) TOTAL AFUCOSYLATED GLYCOFORMS OF ANTIBODIES PRODUCED IN CELL CULTURE

(71) Applicant: AMGEN INC., Thousand Oaks, CA (US)

(72) Inventors: Prince BHEBE, Moorpark, CA (US); Matthew JERUMS, Oak View, CA (US); Irene LIU, Poway, CA (US); Kurt Thomas KUNAS, Westlake Village, CA (US)

(73) Assignee: AMGEN INC., Thousand Oaks, CA (US)

(21) Appl. No.: 19/028,183

(22) Filed: Jan. 17, 2025

Related U.S. Application Data
(62) Division of application No. 17/040,422, filed on Sep. 22, 2020.
(60) Provisional application No. 62/648,308, filed on Mar. 26, 2018.

(51) Int. Cl. C07K 16/00; C07K 16/30; C12P 21/00

(52) U.S. Cl. CPC C07K 16/00; C07K 16/30; C12P 21/005; C07K 2317/14; C07K 2317/41; C07K 2317/51; C07K 2317/515; C07K 2317/732

(57) ABSTRACT

Provided herein are methods of producing an antibody composition comprising a desired or predetermined or pre-selected level of total afucosylated (TAF) glycoforms. In exemplary embodiments, the method comprises maintaining glycosylation-competent cells in a cell culture medium comprising fucose and/or glucose at a specific concentration as described herein, depending on the level of TAF glycoforms desired. Related compositions comprising glycosylated proteins and TAF glycoforms thereof are also provided herein. Also provided are cell culture media.

### CROSS-REFERENCE TO RELATED APPLICATIONS

This application is a divisional of U.S. patent application Ser. No. 17/040,422, filed Sep. 22, 2020, which is a national stage application under 35 U.S.C. § 371 of International Application No. PCT/US2019/024154, having an international filing date of Mar. 26, 2019; which claims the benefit under 35 U.S.C. 119(e) of U.S. Provisional Application No. 62/648,308, filed Mar. 26, 2018, all of which are incorporated by reference herein in their entireties.

### INCORPORATION BY REFERENCE OF MATERIAL SUBMITTED ELECTRONICALLY

Incorporated by reference in its entirety is a computer-readable nucleotide/amino acid sequence listing submitted concurrently herewith and identified as follows: 37.6 KB XML file named “A-2173-US03-DIV.XML”; created on Jan. 16, 2025.

### BACKGROUND

Glycosylation is one of the most common, yet important, post-translational modifications, as it plays a role in multiple cellular functions, including, for example, protein folding, quality control, molecular trafficking and sorting, and cell surface receptor interaction. Glycosylation affects the therapeutic efficacy of recombinant protein drugs, as it influences the bioactivity, pharmacokinetics, immunogenicity, solubility, and in vivo clearance of a therapeutic glycoprotein. Fc glycoform profiles, in particular, are important product quality attributes for recombinant antibodies, as they directly impact the clinical efficacy and pharmacokinetics of the antibodies.

The high mannose (HM) glycoform content has been found to affect pharmacokinetic properties of certain therapeutic antibodies (Goetze, et al., (2011) Glycobiology 21, 949-59; Yu, et al., (2012) MAbs 4, 475-87). HM glycoforms not only influence the serum clearance rate of the antibodies, but such glycoforms, in addition to afucosylated (afuco) glycoforms, can impact antibody effector function or antibody-mediated target cell killing, also known as antibody-dependent cellular cytotoxicity (ADCC).

Many factors influence the glycan structure and thus the ultimate glycosylated form (glycoform) of the protein (glycoprotein). For example, the cell line expressing the antibody, the cell culture medium, the feed medium composition, and the timing of the feeds during cell culture can impact the production of glycoforms of the protein.

While research groups have suggested many ways to influence the levels of particular glycoforms of an antibody, there still is a need in the biopharmaceutical industry for simple and efficient methods to manipulate and control the levels of total afucosylated (TAF) glycoforms during recombinant production of therapeutic antibodies.

### SUMMARY

Described for the first time are data demonstrating that the concentration of fucose and/or glucose in a cell culture medium comprising cells producing a recombinant glycosylated protein (e.g., an antibody or antibody binding protein) influences the level of TAF glycoforms of the recombinant glycosylated protein produced. Whereas larger changes in the level of TAF glycoforms of a recombinant glycosylated protein (e.g., an antibody or antibody binding protein) can be achieved by manipulating the concentration of fucose in the cell culture medium comprising cells producing the recombinant glycosylated protein (e.g., antibody or antibody binding protein), smaller changes in the TAF glycoforms level can be achieved by altering the concentration of glucose in the cell culture medium, as described herein. Also, the data demonstrate that, while glucose concentration of the cell culture medium affects levels of high mannose glycans and afucosylated glycans, the fucose concentration of the cell culture medium affects levels of afucosylated glycans but does not influence high mannose glycan levels. The discovery that each of these sugars, differing in chemical formula by only one oxygen atom, leads to differential effects on TAF glycoform levels was unexpected. Without being bound to a particular theory, maintaining cells producing the recombinant glycosylated protein (e.g., antibody or antibody binding protein) in a cell culture medium comprising fucose and/or glucose at concentrations as taught herein allows for production of a recombinant glycosylated protein (e.g., an antibody or antibody binding protein) composition having a desired or predetermined or pre-selected level of TAF glycoforms (e.g., high mannose glycans and afucosylated glycans). Accordingly, the disclosure relates to methods of producing a recombinant glycosylated protein composition (e.g., an antibody composition or antibody binding protein composition) comprising a desired or predetermined or pre-selected level of TAF glycoforms.

The disclosure provides methods of producing a recombinant glycosylated protein composition (e.g., an antibody composition or antibody binding protein composition). In exemplary embodiments, the method comprises maintaining glycosylation-competent cells in a cell culture medium comprising fucose and/or glucose at a specific concentration as described herein, depending on the level of TAF glycoforms desired.

In exemplary embodiments, the level of TAF glycoforms in the recombinant glycosylated protein composition (e.g., antibody composition or antibody binding protein composition) is less than or about 10% and, in exemplary aspects, the method comprises maintaining glycosylation-competent cells in a cell culture medium comprising fucose, wherein fucose is present in the culture medium at a concentration between about 0.17 g/L and about 1.0 g/L.

In exemplary embodiments, the level of TAF glycoforms in the recombinant glycosylated protein composition (e.g., antibody composition or antibody binding protein composition) is less than or about 10% and, in exemplary aspects, the method comprises maintaining glycosylation-competent cells in a cell culture medium comprising fucose, wherein fucose is present in the culture medium at a concentration between about 0.1 g/L and about 1.0 g/L, and wherein the glycosylation-competent cells are not genetically modified to alter activity of an enzyme of the de novo pathway or the salvage pathway.

The disclosure also provides methods of producing a recombinant glycosylated protein composition (e.g., an antibody composition or antibody binding protein composition) comprising maintaining glycosylation-competent cells in a cell culture medium comprising fucose and glucose, wherein fucose is present in the culture medium at a concentration of about 0.1 g/L to about 1.0 g/L and adding glucose to the cell culture medium according to a glucose feeding schedule that achieves an average glucose concentration of about 10 g/L or less.

Recombinant glycosylated protein compositions (e.g., antibody compositions or antibody binding protein compositions) produced by the methods of the disclosure are provided herein. Additionally, related pharmaceutical compositions and cell culture media are provided. In exemplary aspects, the cell culture medium comprises comprising an exogenous nucleic acid encoding an antibody (e.g., an IgG antibody) and a culture medium comprising fucose at a concentration of about 0.1 g/L to about 1.0 g/L or about 0.17 g/L to about 1.0 g/L. In some instances, the glycosylation-competent cells are not genetically modified to alter activity of an enzyme of the de novo pathway or the salvage pathway. Optionally, the glycosylation-competent cells are not genetically modified to knock-out a gene encoding GDP-keto-6-deoxymannonse-3,5-epimerase, 4-reductase. The culture medium, in some aspects, further comprises glucose at a concentration less than about 10 g/L, optionally, less than about 9 g/L or about 6 g/L or less (e.g., about 0.5 g/L to about 4 g/L). In exemplary instances, the pH of the culture medium is about 6.85 to about 7.05, e.g., about 6.90 to about 7.00. In some instances, the cell culture medium does not comprise mannose. In certain aspects, the antibody is an IgG1 antibody. In exemplary aspects, the antibody is specific for a tumor-associated antigen, such as one comprising SEQ ID NO: 3.

Methods of altering or modulating the level of TAF glycans of a recombinant glycosylated protein composition (e.g., an antibody composition or antibody binding protein composition) produced by glycosylation-competent cells in a cell culture medium are further provided herein. In exemplary aspects, the method comprises (A) adding fucose to a cell culture medium comprising the glycosylation-competent cells to achieve a fucose concentration of about 0.1 g/L to about 1.0 g/L to decrease the level of TAF glycans; (B) adding glucose to a cell culture medium comprising the glycosylation-competent cells to achieve a glucose concentration less than about 10 g/L to increase the level of TAF; or (C) a combination of both (A) and (B).

Also provided are methods of modulating the level of afucosylated glycans of a recombinant glycosylated protein composition (e.g., an antibody composition or antibody binding protein composition) produced by glycosylation-competent cells. In exemplary embodiments, the method comprises (A) adding fucose to a cell culture medium comprising the glycosylation-competent cells to achieve a fucose concentration of about 0.1 g/L to about 1.0 g/L, to decrease the level of afucosylated glycans; (B) adding glucose to a cell culture medium comprising the glycosylation-competent cells to achieve a glucose concentration less than or about 10 g/L to increase the level of afucosylated glycans; or (C) a combination of both (A) and (B).

The present disclosure further provides a method of modulating the level of high mannose (HM) glycans of a recombinant glycosylated protein composition (e.g., an antibody composition or antibody binding protein composition) produced by glycosylation-competent cells. In exemplary embodiments, the method comprises adding glucose to a cell culture medium comprising the glycosylation-competent cells to achieve a glucose concentration less than about 10 g/L to increase the level of HM glycans.

Also provided by the present disclosure are methods of modulating the level of afucosylated glycans of a recombinant glycosylated protein composition (e.g., an antibody composition or antibody binding protein composition) produced by glycosylation-competent cells comprising reducing the pH of the cell culture medium by about 0.03 to about 1.2 to reduce the level of afucosylated glycans of the composition by about 0.5% to about 2% or increasing the pH of the cell culture medium by about 0.03 to about 1.2 to increase the level of afucosylated glycans of the composition by about 0.5% to about 2%.

Methods of reducing the level of afucosylated glycans of a recombinant glycosylated protein composition (e.g., an antibody composition or antibody binding protein composition) produced by glycosylation-competent cells by about 1% to about 2%, comprising reducing the pH of the cell culture medium by about 0.05 to about 1.2 are provided by the present disclosure.

Additionally provided are methods of reducing the level of afucosylated glycans of a recombinant glycosylated protein composition (e.g., an antibody composition or antibody binding protein composition) produced by glycosylation-competent cells by about 0.5% to about 1.1%, comprising reducing the pH of the cell culture medium by about 0.03-0.07.

The present disclosure further provides methods of increasing the level of afucosylated glycans of a recombinant glycosylated protein composition (e.g., an antibody composition or antibody binding protein composition) produced by glycosylation-competent cells by about 1% to about 2%, comprising increasing the pH of the cell culture medium by about 0.05 to about 1.2.

Also provided are methods of increasing the level of afucosylated glycans of a recombinant glycosylated protein composition (e.g., an antibody composition or antibody binding protein composition) produced by glycosylation-competent cells by about 0.5% to about 1.1%, comprising increasing the pH of the cell culture medium by about 0.03-0.07.

The present disclosure further provides methods of modulating the level of TAF glycans of a recombinant glycosylated protein composition (e.g., an antibody composition or antibody binding protein composition) produced by glycosylation-competent cells, comprising modulating, reducing or increasing, the level of afucosylated glycans of the composition in accordance with a presently disclosed method of modulating, reducing or increasing, the level of afucosylated glycans.

The present disclosure provides a method of producing a recombinant glycosylated protein composition (e.g., an antibody composition or antibody binding protein composition), wherein the level of afucosylated glycans in the composition is about 6.2% to about 8.4%, the method comprising maintaining glycosylation-competent cells in a cell culture medium at a pH higher than 7.05 and lower than 7.2, wherein: (A) the pH of the cell culture medium changes by less than 0.15 (optionally by less than 0.10) during the culture period or (B) the temperature of the cell culture medium changes by not more than 2 degrees C. or the method does not comprise culturing the cells in a cell culture medium comprising manganese or betaine or (D) a combination of two or three of (A), (B), and (C).

### BRIEF DESCRIPTION OF THE DRAWINGS

FIG. 1A is an illustration of the three types of N-glycans (oligomannose, complex and hybrid) and commonly used symbols for such saccharides.

FIG. 1B is an illustration of exemplary glycan structures.

FIG. 2 is a diagram of the salvage pathway and the de novo pathway of fucose metabolism. In the salvage pathway, free L-fucose is converted to GDP-fucose, while in the de novo pathway, GDP-fucose is synthesized via three reactions catalyzed by GMD and FX. GDP-fucose is then transported from the cytosol to the Golgi lumen by GDP-Fuc Transferase and transferred to acceptor oligosaccharides and proteins. The other reaction product, GDP, is converted by a luminal nucleotide diphosphatase to guanosine 5-monophosphate (GMP) and inorganic phosphate (Pi). The former is exported to the cytosol (via an antiport system that is coupled with the transport of GDP-fucose), whereas the latter is postulated to leave the Golgi lumen via the Golgi anion channel, GOLAC. See, e.g., Nordeen et al. 2000; Hirschberg et al. 2001.

FIG. 3 is a graph depicting (A) the glucose concentration (g/L) of the cell culture using a control medium (line with open triangles), a first test medium (line with open circles), and a second test medium (line with open hexagons), over the course of the cell culture run, (B) the fucose concentration (g/L) of the cell culture using the second test medium (line with open squares) over the cell culture run, and (C) the TAF glycan levels (%) of the cell culture using the control medium (dotted line with closed triangles), the first test medium (dotted line with closed circles), and second test medium (dotted line with closed hexagons), over the course of the cell culture run.

FIG. 4 is a graph depicting (A) the glucose concentration (g/L) of the cell culture using a control medium (line with open triangles), a first test medium (line with open circles), and a second test medium (line with open hexagons), over the course of the cell culture run, and (B) the high mannose (HM) glycan levels (%) of the cell culture using the control medium (dotted line with closed triangles), the first test medium (dotted line with closed circles), and second test medium (dotted line with closed hexagons), over the course of the cell culture run.

FIG. 5 is a graph depicting (A) the glucose concentration (g/L) of the cell culture using a control medium (line with open triangles), a first test medium (line with open circles), and a second test medium (line with open hexagons), over the course of the cell culture run, and (B) the afucosylated (afuc) glycan levels (%) of the cell culture using the control medium (dotted line with closed triangles), the first test medium (dotted line with closed circles), and second test medium (dotted line with closed hexagons), over the course of the cell culture run.

FIG. 6 is a graph of the TAF glycan levels (%) as a function of fucose concentration (g/L) in the cell culture medium containing 0× glucose, 1× glucose or 2× glucose.

FIG. 7 is a graph of the ADCC levels (expressed as % relative to a control antibody having the same amino acid sequence) as a function of fucose concentration (g/L) in the cell culture medium containing 0× glucose, 1× glucose or 2× glucose.

FIG. 8 is a graph illustrating a model of the effects of glucose and fucose on TAF glycan levels (%). The min, max, and mean TAF according to the QTPP are shown. The equation below the graph shows the mathematical relationship between glucose, fucose and TAF.

FIG. 9A is a series of graphs showing: (i) TAF glycan levels (%) as a function of fucose concentration (g/L) in the cell culture medium (top left quadrant) or as a function of glucose concentration (g/L) in the cell culture medium (top right quadrant) and (ii) ADCC levels (expressed as % relative to a control antibody having the same amino acid sequence) as a function of fucose concentration (g/L) in the cell culture medium (bottom left quadrant) or as a function of glucose concentration (g/L) in the cell culture medium (bottom right quadrant). The range of TAF glycan levels (%) is 3.30684 to 3.73083 and the range of ADCC levels is 78.3092-90.4408. At 0.2 g/L fucose and 3.0 g/L glucose, the TAF glycan level (%) was 3.518836, and the ADCC level (%) was 84.37501.

FIG. 9B is a series of graphs showing: (i) TAF glycan levels (%) as a function of fucose concentration (g/L) in the cell culture medium (top left quadrant) or as a function of glucose concentration (g/L) in the cell culture medium (top right quadrant) and (ii) ADCC levels (expressed as % relative to innovator or commercially-available antibody) as a function of fucose concentration (g/L) in the cell culture medium (bottom left quadrant) or as a function of glucose concentration (g/L) in the cell culture medium (bottom right quadrant). The range of TAF glycan levels (%) is 3.52301 to 4.0559 and the range of ADCC levels is 75.6911-90.9385. At 0 g/L fucose and 0.554 g/L glucose, the TAF glycan level (%) was 3.789458, and the ADCC level (%) was 83.3148.

FIG. 9C is a series of graphs showing: (i) TAF glycan levels (%) as a function of fucose concentration (g/L) in the cell culture medium (top left quadrant) or as a function of glucose concentration (g/L) in the cell culture medium (top right quadrant) and (ii) ADCC levels (expressed as % relative to innovator or commercially-available antibody) as a function of fucose concentration (g/L) in the cell culture medium (bottom left quadrant) or as a function of glucose concentration (g/L) in the cell culture medium (bottom right quadrant). The range of TAF glycan levels (%) is 2.9975-3.68468 and the range of ADCC levels is 79.2215-98.8836. At 0.492 g/L fucose and 6.0 g/L glucose, the TAF glycan level (%) was 3.341092, and the ADCC level (%) was 89.05256.

FIG. 10 is a graph of osmolality plotted as a function of fucose concentration.

FIG. 11 is a graph of fucose concentration plotted as a function of time (duration) in cell culture.

FIG. 12 is a graph of the % TAF and fucose feed, each plotted as a function of time.

FIG. 13 is a pair of graphs demonstrating that controlling glucose in the target range from Day 6 or earlier yields equivalent TAF results.

FIG. 14 is a graph of % afucosylation plotted as a function of culture time.

### DETAILED DESCRIPTION

Many secreted proteins undergo post-translational glycosylation, a process by which sugar moieties (e.g., glycans, saccharides) are covalently attached to specific amino acids of a protein. In eukaryotic cells, two types of glycosylation reactions occur: (1) N-linked glycosylation, in which glycans are attached to the asparagine of the recognition sequence Asn-X-Thr/Ser, where “X” is any amino acid except proline, and (2) O-linked glycosylation in which glycans are attached to serine or threonine. Regardless of the glycosylation type (N-linked or O-linked), microheterogeneity of protein glycoforms exists due to the large range of glycan structures associated with each site (O or N).

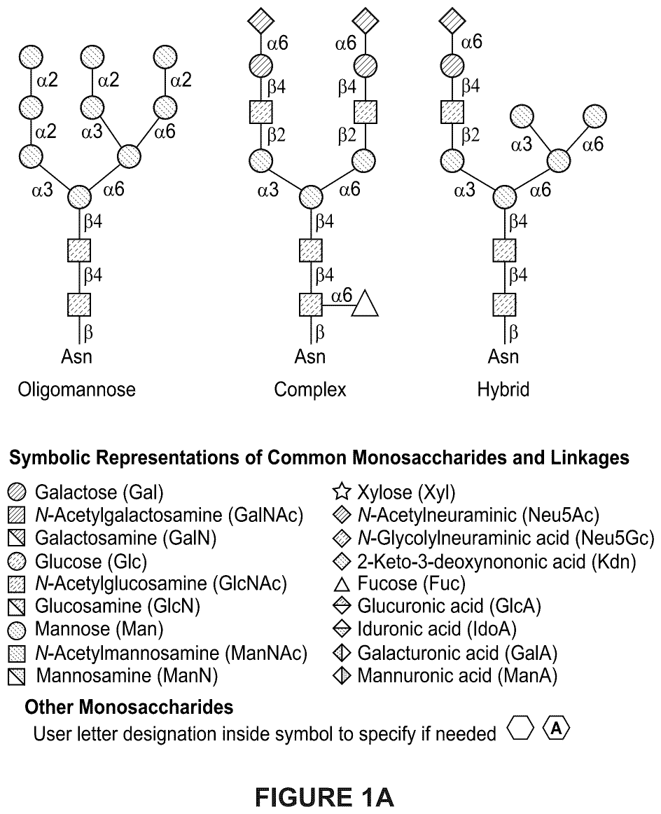

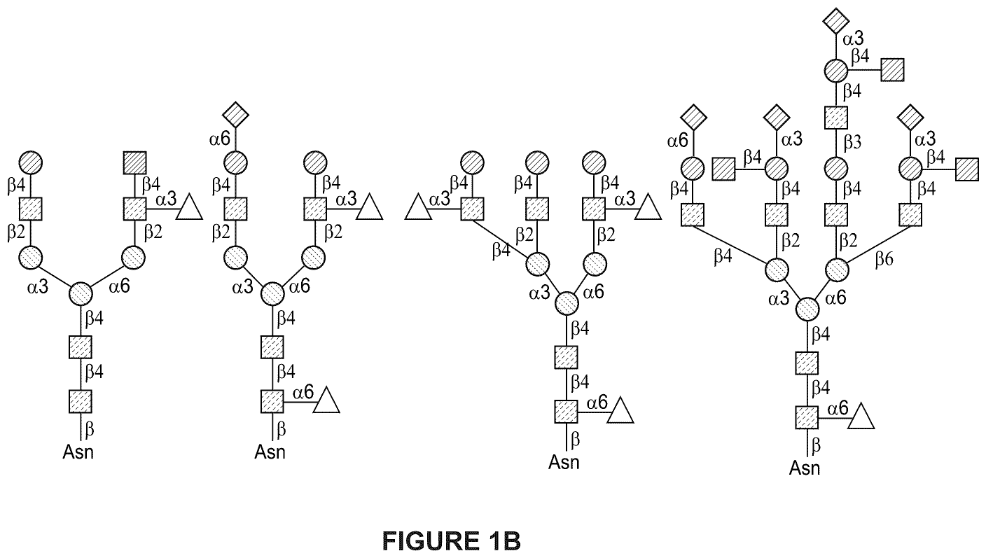

All N-glycans have a common core sugar sequence: Manα1-6(Manα1-3)Manβ1-4GlcNAcβ1-4GlcNAcβ1-Asn-X-Ser/Thr (Man₃GlcNAc₂Asn) and are categorized into one of three types: (A) a high mannose (HM) or oligomannose (OM) type, which consists of two N-acetylglucosamine (GalNAc) moieties and a large number (e.g., 5, 6, 7, 8 or 9) of mannose (Man) residues (B) a complex type, which comprises more than two GlcNAc moieties and any number of other sugar types or (C) a hybrid type, which comprises a Man residue on one side of the branch and GlcNAc at the base of a complex branch. FIG. 1A (taken from Stanley et al., Chapter 8: N-Glycans, Essentials of Glycobiology, 2^{nd} ed., Cold Spring Harbor Laboratory Press; 2009) shows the three types of N-glycans.

N-linked glycans typically comprise one or more monosaccharides of galactose (Gal), N-acetylgalactosamine (GalNAc), galactosamine (GalN), glucose (GLc), N-acetylglucosamine (ClcNAc), glucoasamine (GlcN), mannose (Man), N-Acetylmannosamine (ManNAc), Mannosamine (ManN), xylose (Xyl), N0Acetylneuraminic acid (Neu5Ac), N-Glycolylneuraminic acid (Neu5Gc), 2-keto-3-doxynononic acid (Kdn), fucose (Fuc), Glucuronic acid (GLcA), Iduronic acid (IdoA), Galacturonic acid (Gal A), mannuronic acid (Man A). The commonly used symbols for such saccharides are shown in FIG. 1A. Exemplary glycans and their identity are shown in FIG. 1B.

N-linked glycosylation begins in the endoplasmic reticulum (ER), where a complex set of reactions result in the attachment of a core glycan structure made essentially of two GlcNAc residues and three Man residues. The glycan complex formed in the ER is modified by action of enzymes in the Golgi apparatus. If the saccharide is relatively inaccessible to the enzymes, it typically stays in the original HM form. If enzymes can access the saccharide, then many of the Man residues are cleaved off and the saccharide is further modified, resulting in the complex type N-glycans structure. For example, mannosidase-1 located in the cis-Golgi, can cleave or hydrolyze a HM glycan, while fucosyltransferase FUT-8, located in the medial-Golgi, fucosylates the glycan (Hanrue Imai-Nishiya (2007), BMC Biotechnology, 7:84).

Accordingly, the sugar composition and the structural configuration of a glycan structure varies, depending on the glycosylation machinery in the ER and the Golgi apparatus, the accessibility of the machinery enzymes to the glycan structure, the order of action of each enzyme and the stage at which the protein is released from the glycosylation machinery, among other factors.

The disclosure provided herein relates to methods of producing an antibody composition comprising a desired or predetermined or pre-selected level of TAF glycoforms. In exemplary embodiments, the method comprises maintaining glycosylation-competent cells in a cell culture medium comprising fucose and/or glucose at a specific concentration as described herein, depending on the level of TAF glycoforms desired. Also, the disclosure relates to method of producing an antibody composition comprising a desired or predetermined or pre-selected level of afucosylated glycoforms, e.g., the level of afucosylated glycans in the antibody composition is about 6.2% to about 8.4%. In exemplary embodiments, the method comprises maintaining glycosylation-competent cells in a cell culture medium at a pH higher than 7.05 and lower than 7.2, wherein: (A) the pH of the cell culture medium changes by less than 0.15 (optionally by less than 0.10) during the culture period or (B) the temperature of the cell culture medium changes by not more than 2 degrees C. or the method does not comprise culturing the cells in a cell culture medium comprising manganese or betaine or (D) a combination of two or three of (A), (B), and (C). Without being bound to a particular theory, it is believed that the methods of the disclosure provide a means for tailor-made compositions comprising specific amounts of particular glycoforms of a given antibody.

In exemplary embodiments, the levels of TAF glycans are modulated. As used herein, “total afucosylated glycans” or “TAF glycans” or “total afucosylated glycoforms” or “TAF glycoforms” refers to the sum amount of high mannose (HM) glycans and afucosylated glycans. In exemplary embodiments, the levels of HM glycans are modulated. As used herein, the term “high mannose glycans” or “HM glycans” or “high mannose glycoforms” or “HM glycofroms” or “HM” encompasses glycans comprising 5, 6, 7, 8, or 9 mannose residues, abbreviated as Man5, Man6, Man7, Man8, and Man9, respectively. In exemplary embodiments, the levels of afucosylated glycans are modulated. As used herein, the term “afucosylated glycan” or “afuco glycan” or “afucosylated glycoform” or “Afuc” refers to glycoforms which lack a core fucose, e.g., an α1,6-linked fucose on the GlcNAc residue involved in the amide bond with the Asn of the N-glycosylation site. Afucosylated glycoforms include, but are not limited to, A1G0, A2G0, A2G1a, A2G1b, A2G2, and A1G1M5. Additional afucosylated glycans include, e.g., A1G1a, G0[H3N4], G0[H4N4], G0[H5N4], FO-N[H3N3]. See, e.g., Reusch and Tejada, Glycobiology 25(12): 1325-1334 (2015). In exemplary aspects, the level of TAF and the amounts of HM glycoforms and afucosylated glycoforms are determined via Hydrophilic Interaction Liquid Chromatography (HILIC), as further described herein in Example 1. After enzyme cleavage of the N-glycans, HILIC is performed to obtain a chromatogram with several peaks, each peak of which represents a mean distribution (amount) of a different glycoform. For these purposes, % Peak Area=Peak Area/Total Peak Area×100%, and % Total Peak Area=Sample Total Area/Total Area of the Standard×100%. The calculations used for purposes of determining the % TAF may be carried out as follows:

$$\% Afucosylated glycoforms = \% A 1 G 0 + \% A 2 G 0 + \% A 2 G 1 a + \% A 2 G 1 b + \% A 2 G 2 + \% A 1 G 1 M 5.$$

$$\% High mannose glycoforms = \% Man 5 ( if detectable ) + \% Man 6 ( if detectable ) + \% Man 7 ( if detectable ) + \% Man 8 ( if detectable ) + \% Man 9 ( if detectable )$$

The disclosure provides methods of producing a recombinant glycosylated protein composition. In exemplary embodiments, the recombinant glycosylated protein composition is an antibody composition. In exemplary embodiments, the method comprises maintaining glycosylation-competent cells in a cell culture medium comprising fucose and/or glucose at a specific concentration as described herein, depending on the level of TAF glycoforms desired.

### Fucose

In exemplary embodiments of the methods disclosed herein, fucose is present in the culture medium at a concentration from about 0.1 g/L to about 2.0 g/L, optionally, about 0.1 g/L to about 1.75 g/L, about 0.1 g/L to about 1.5 g/L, or about 0.1 g/L to about 1.2 g/L. In exemplary instances, fucose is present in the culture medium at a concentration less than or about 1.2 g/L. In exemplary instances, fucose is present in the culture medium at a concentration from about 0.1 g/L to about 1.0 g/L. In exemplary instances, the culture medium comprises fucose at a concentration from about 0.17 g/L to about 2.0 g/L, about 0.17 g/L to about 1.75 g/L, about 0.17 g/L to about 1.5 g/L, or about 0.17 g/L to about 1.2 g/L. In exemplary aspects, fucose is present in the culture medium at a concentration from about 0.17 g/L to about 1.2 g/L. In exemplary instances, fucose is present in the culture medium at a concentration from about 0.17 g/L to about 1.0 g/L. In exemplary instances, the culture medium comprises fucose at a concentration from about 0.2 g/L to about 2.0 g/L, about 0.2 g/L to about 1.75 g/L, about 0.2 g/L to about 1.5 g/L, or about 0.2 g/L to about 1.2 g/L. In exemplary instances, fucose is present in the culture medium at a concentration from about 0.2 g/L to about 1.0 g/L. In exemplary instances, fucose is present in the culture medium at a concentration less than or about 1.0 g/L. For example, in some instances, the fucose concentration of the culture medium is about 0.10 g/L, about 0.11 g/L, about 0.12 g/L, about 0.13 g/L, about 0.14 g/L, about 0.15 g/L, about 0.16 g/L, about 0.17 g/L, about 0.18 g/L, about 0.19 g/L, or about 0.20 g/L. In some instances, the fucose concentration of the culture medium is about 0.3 g/L, about 0.4 g/L, about 0.5 g/L, about 0.6 g/L, about 0.7 g/L, about 0.8 g/L, or about 0.9 g/L. In exemplary aspects, the fucose concentration is not more than or about 1.0 g/L, not more than or about 0.9 g/L, not more than or about 0.8 g/L, or not more than or about 0.7 g/L. In exemplary instances, fucose is present in the culture medium at a concentration less than or about 0.75 g/L, or about 0.25 g/L to about 0.75 g/L, e.g., about 0.4 g/L to about 0.5 g/L, or about 0.6 g/L. In exemplary aspects, fucose is present in the culture medium at a concentration of less than about 0.6 g/L, e.g., about 0.2 g/L to about 0.5 g/L.

In exemplary aspects, the method of producing a recombinant glycosylated protein composition (e.g., an antibody composition or antibody binding protein composition) comprises maintaining the glycosylation-competent cells in two different cell culture media. In exemplary aspects, the method of producing a recombinant glycosylated protein composition (e.g., an antibody composition or antibody binding protein composition) comprises maintaining the glycosylation-competent cells in a first cell culture medium for an initial time period and subsequently maintaining the glycosylation-competent cells in a second cell culture medium, optionally, wherein the first cell culture medium does not comprise fucose at a concentration of about 0.1 g/L to about 1.0 g/L and the second cell culture medium comprises fucose at a concentration of about 0.1 g/L to about 1.0 g/L and the second cell culture medium comprises fucose, e.g., at one of the above concentrations. In exemplary instances, the initial time period begins when cells are inoculated into a bioreactor comprising cell culture medium, e.g., the cell culture medium. In some aspects, the initial time period is about 1 day to about 3 days, e.g., about 24 hours to about 72 hours. In exemplary aspects, the initial time period is greater than about 3 days (about 72 hours) but less than about 10 days (about 240 hours) or less than about 156 hours. In exemplary aspects, the initial time period is about 3, about 4, about 5, about 6, about 7, about 8, or about 9 days. In exemplary aspects, the method comprises adding fucose to the culture medium after the initial time period. In some aspects, fucose is added to the first culture medium to obtain the second cell culture medium. For example, in various aspects, the method comprises adding fucose after about 1 day to about 3 days, after about 3 days but less than about 10 days, or after about 3, about 4, about 5, about 6, about 7, about 8, or about 9 days. In exemplary aspects, the method comprises adding fucose to the cell culture medium, e.g., the first cell culture medium, on the 6th day, 7^{th} day, 8^{th} day, or 9^{th} day post-cell culture inoculation. In exemplary aspects, fucose is added to a final concentration greater than about 0.1 g/L, greater than about 0.17 g/L, or greater than about 0.2 g/L, and less than about 2.0 g/L. In exemplary aspects, the first cell culture medium does not comprise fucose. In exemplary aspects, the first cell culture medium comprises fucose, but at a concentration that is undetectable or immeasurable, or at a concentration that is substantially below the fucose concentration of the second cell culture medium, e.g., substantially below 0.1 g/L, below about 0.17 g/L, or below about 0.2 g/L.

In alternative aspects, the methods comprising maintaining glycosylation-competent cells in a cell culture medium comprising fucose (e.g., at a concentration greater than about 0.1 g/L, greater than about 0.17 g/L, or greater than about 0.2 g/L, and less than about 2.0 g/L) for the entire duration the glycosylation-competent cells are maintained in cell culture, or for a large part of the culture period. In some aspects, the method of producing a recombinant glycosylated protein composition (e.g., an antibody composition or antibody binding protein composition) comprises inoculating the glycosylation-competent cells in a bioreactor comprising cell culture medium comprising fucose and maintaining the cells in the cell culture medium at the concentration of fucose is maintained to be substantially the same throughout the duration of the cell culture.

In exemplary embodiments, the concentration of fucose fluctuates very little during the course of cell culture. In exemplary aspects, the fucose concentration fluctuates by about 0.2 g/L or less during the time the glycosylation-competent cells are maintained in the cell culture medium comprising fucose. In exemplary aspects, the concentration of fucose fluctuates by about 0.1 g/L or less during the time the glycosylation-competent cells are maintained in the cell culture medium comprising fucose. In exemplary aspects, when fucose is added to the cell culture medium, e.g., after the initial cell culture period, fucose is added to the medium not more than once or twice during the cell culture period.

### Glucose

In exemplary embodiments of the methods disclosed herein, glucose is present in the culture medium. In exemplary aspects, glucose is present in the culture medium at a concentration less than or about 10 g/L, less than or about 9.0 g/L, or less than or about 6.0 g/L. In exemplary aspects, glucose is present in the culture medium at a concentration from about 0.5 g/L to about 4.0 g/L. In exemplary aspects, glucose is present at a concentration from about X g/L to about Y g/L, wherein X is about 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, or 3.9, and Y is about 0.6, 0.7, 0.8, 0.9, 1.0, 1.1, 1.2, 1.3, 1.4, 1.5, 1.6, 1.7, 1.8. 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0, provided that X is less than Y.

In exemplary aspects, the method comprises maintaining the concentration of the glucose in the cell culture medium for a time period that is equivalent to the cell culture period. In exemplary instances, maintaining the concentration of the glucose in the cell culture medium comprises sampling the cell culture medium on a regular basis, e.g., hourly, bi-hourly, once every 3, 4, 5, or 6 hours, once a day, twice a day, three times daily, or 4 times daily, and the like, measuring the glucose concentration of the sampled cell cultured medium, and adding glucose to the cell culture, if the glucose concentration of the sampled cell cultured medium is lower than the desired maintained glucose concentration. In exemplary aspects, maintaining the concentration of the glucose in the cell culture medium comprises measuring the glucose concentration of the cell culture medium via a glucose sensor. In exemplary aspects, the glucose concentration is measured via a glucose sensor at regular intervals, e.g., hourly, bi-hourly, once every 3, 4, 5, or 6 hours, once a day, twice a day, three times daily, or 4 times daily, and the like, and glucose is added to the cell culture, if the glucose concentration is determined via the glucose sensor to be lower than the desired maintained glucose concentration. In alternative aspects, the method comprises maintaining the glycosylation-competent cells in cell culture medium comprising glucose, but maintaining the concentration of the glucose in the cell culture medium only after an initial time period. In exemplary embodiments, the initial time period is about 1 day to about 3 days (about 24 hours to about 72 hours). In some instances, the initial time period is less than or about 6 days, optionally, wherein the initial time period is 3 days or 4 days or 5 days after cell culture inoculation. In exemplary aspects, the method comprises maintaining the glycosylation-competent cells in cell culture medium comprising glucose and maintaining the concentration of the glucose in the cell culture medium on the 6^{th} day post-inoculation and subsequently thereafter. In exemplary aspects, the concentration of glucose is maintained for at least about 4 days or about 5 days following the initial time period, or optionally, maintaining for at least about 6 days following the initial time period.

In exemplary aspects, the cell culture medium comprises an initial glucose concentration for an initial time period. For example, in various aspects, the initial glucose concentration is about 1.0 g/L to about 15 g/L, about 1.0 to about 12 g/L, or about 1.0 g/L to about 10 g/L. The initial glucose concentration, in some aspects, is about 1.0 g/L, about 1.5 g/L, about 2.0 g/L, about 2.5 g/L, about 3.0 g/L, about 3.5 g/L, about 4.0 g/L, about 4.5 g/L, about 5.0 g/L, about 5.5 g/L, about 6.0 g/L, about 6.5 g/L, about 7.0 g/L, about 7.5 g/L, about 8.0 g/L, about 8.5 g/L, about 9.0 g/L, about 9.5 g/L, about 10.0 g/L, about 10.5 g/L, about 11.0 g/L, about 11.5 g/L, or about 12.0 g/L. In some aspects, the initial glucose concentration is about 12 g/L±1 g/L or about 9 g/L±1 g/L or about 6 g/L±1 g/L. In some aspects, the initial glucose concentration is less than about 5.0 g/L or less than about 4.0 g/L. In exemplary aspects, the initial glucose concentration is the glucose concentration of the cell culture medium used during the initial time period. In exemplary aspects, the initial glucose concentration is the glucose concentration of the cell culture medium maintained during the initial time period.

In exemplary aspects, the initial glucose concentration is the same as the glucose concentration maintained after the initial time period. In alternative aspects, the initial glucose concentration is different from the glucose concentration maintained after the initial time period. In exemplary aspects, the method comprises adding glucose to the cell culture medium after the initial time period and maintaining glucose at a different concentration relative to the initial glucose concentration. In exemplary aspects, the method comprises adding glucose to the cell culture medium after the initial time period to maintain glucose at a different concentration relative to the initial glucose concentration, wherein the step of adding glucose achieves a glucose concentration of about 10 g/L or less (e.g., about 9 g/L or less, about 6 g/L or less, about 0.5 g/L to about 4 g/L).

In exemplary aspects, the method comprises adding glucose to the cell culture medium according to a glucose feeding schedule. In some aspects, the glucose feeding schedule is initiated after the initial time period. For example, in some aspects, the initial time period is at least 3 days or 4 days and the glucose feeding schedule is initiated about 4 to about 6 days post-cell culture inoculation, e.g., about 4 days, about 5 days, about 6 days post-cell culture inoculation. In exemplary instances, the glucose feeding schedule achieves an average glucose concentration of about 10 g/L or less (e.g., about 9 g/L or less, about 6 g/L or less, about 0.5 g/L to about 4 g/L). The term “average glucose concentration” refers to the average concentrations of glucose in the cell culture medium as determined by a glucose sensor over a time period (e.g., 1 to 2 days). In exemplary instances, the glucose feeding schedule achieves an average glucose concentration based on the concentration of fucose of the cell culture medium. In some aspects, the average glucose concentration is calculated based on Formula I:

$$T = 3.354 - 1.388 F + 0.111 G + [ F - 0.43 \; 75 ] \times [ 1.9527 ( F - 0.4375 ) ] \qquad ( Formula 1 )$$

wherein T is the targeted % total afucosylated (TAF) glycans and is about 2.5% to about 6%, about 2.75% to about 5.5%, or about 3% to about 5%, F is the concentration (g/L) of fucose in the medium, and G is the average glucose concentration (g/L).

In exemplary instances, (i) the concentration of fucose is about 0.2±0.1 g/L and the average glucose concentration is about 2 to about 4 g/L; (ii) the concentration of fucose is about 0.5±0.1 g/L and the average glucose concentration is about 3 to about 6 g/L; or (iii) the concentration of fucose is about 0.75±0.1 g/L and the average glucose concentration is about 4.5 to about 9 g/L.

### TAF, HM, and Afucosylated Glycan Levels

In exemplary embodiments, the methods disclosed herein produce a recombinant glycosylated protein composition (e.g., an antibody composition or antibody binding protein composition), wherein the level of TAF glycans in the composition is less than or about 10%. In exemplary aspects, the level of TAF glycans in the composition is less than or about 9%, less than or about 8%, less than or about 7%, less than or about 6%, less than or about 5%. In exemplary aspects, the level of TAF glycans in the composition is greater than or about 4%, e.g., between about 4% and about 10%. In some aspects, the level of TAF glycans in the composition is about 2% to about 6% or about 2.5% to about 5%. In some aspects, the level of TAF glycans is about 2.0%, about 2.5%, about 3.0%, about 3.5%, about 4.0%, about 4.5%, about 5%, about 5.5%, or about 6.0%. In exemplary aspects, the level of TAF glycans is about 2% to about 5% or about 2% to about 4%.

In exemplary aspects of the methods of producing a recombinant glycosylated protein composition (e.g., an antibody composition or antibody binding protein composition), fucose is present in the culture medium at a concentration between about 0.1 g/L and about 1.0 g/L, or between about 0.17 g/L and about 1.0 g/L, and the level of TAF glycans in the composition is less than about 10%.

In exemplary embodiments, the methods disclosed herein produce a recombinant glycosylated protein composition (e.g., an antibody composition or antibody binding protein composition), wherein the level of high mannose glycans in the antibody composition is less than or about 3.5%, e.g., less than or about 3.25%, less than or about 3.0%, less than or about 2.5%, less than or about 2.0%. In exemplary aspects, the level of high mannose glycans in the antibody composition is about 0.7% to about 3.0%, optionally, about 0.7%, about 0.8%, about 0.9%, about 1.0%, about 1.1%, about 1.2%, about 1.3%, about 1.4%, about 1.5%, about 1.6%, about 1.7%, about 1.8%, about 1.9%, about 2.0%, about 2.1%, about 2.2%, about 2.3%, about 2.4%, about 2.5%, about 2.6%, about 2.7%, about 2.8%, about 2.9%, or about 3.0%.

In exemplary embodiments, the methods disclosed herein produce a recombinant glycosylated protein composition (e.g., an antibody composition or antibody binding protein composition), wherein the level of afucosylated glycans in the antibody composition is less than or about 3.5%, e.g., less than or about 3.25%, less than or about 3.0%, less than or about 2.5%, less than or about 2.0%. In exemplary aspects, the level of afucosylated glycans in the antibody composition is about 0.8% to about 2.8%, optionally, about 0.8%, about 0.9%, about 1.0%, about 1.1%, about 1.2%, about 1.3%, about 1.4%, about 1.5%, about 1.6%, about 1.7%, about 1.8%, about 1.9%, about 2.0%, about 2.1%, about 2.2%, about 2.3%, about 2.4%, about 2.5%, about 2.6%, about 2.7%, or about 2.8%.

### Methods of Glycoform Measurement

Various methods are known in the art for assessing glycoforms present in a glycoprotein-containing composition or for determining, detecting or measuring a glycoform profile of a particular sample comprising glycoproteins. Suitable methods include, but are not limited to, positive ion MALDI-TOF analysis, negative ion MALDI-TOF analysis, weak anion exchange (WAX) chromatography, normal phase chromatography (NP-HPLC), exoglycosidase digestion, Bio-Gel P-4 chromatography, anion-exchange chromatography and one-dimensional n.m.r. spectroscopy, and combinations thereof. See, e.g., Mattu et al., JBC 273: 2260-2272 (1998); Field et al., Biochem J 299(Pt 1): 261-275 (1994); Yoo et al., MAbs 2(3): 320-334 (2010) Wuhrer M. et al., Journal of Chromatography B, 2005, Vol.825, Issue 2, pages 124-133; Ruhaak L. R., Anal Bioanal Chem, 2010, Vol. 397:3457-3481 and Geoffrey, R. G. et. al. Analytical Biochemistry 1996, Vol. 240, pages 210-226. Also, the examples set forth herein describe a suitable method for assessing glycoforms present in a glycoprotein containing composition.

With regard to the disclosure, the cell culture may be maintained according to any set of conditions suitable for a recombinant glycosylated protein production. For example, in some aspects, the cell culture is maintained at a particular pH, temperature, cell density, culture volume, dissolved oxygen level, pressure, osmolality, and the like. In exemplary aspects, the cell culture prior to inoculation is shaken (e.g., at 70 rpm) at 5% CO₂ under standard humidified conditions in a CO₂ incubator. In exemplary aspects, the cell culture is inoculated with a seeding density of about 106 cells/mL in 1.5 L medium.

In exemplary aspects, the methods of the disclosure comprise maintaining the glycosylation-competent cells in a cell culture medium at a pH of about 6.85 to about 7.05, e.g., in various aspects, about 6.85, about 6.86, about 6.87, about 6.88, about 6.89, about 6.90, about 6.91, about 6.92, about 6.93, about 6.94, about 6.95, about 6.96, about 6.97, about 6.98, about 6.99, about 7.00, about 7.01, about 7.02, about 7.03, about 7.04, or about 7.05. In some aspects, the cell culture medium has a pH of about 6.9 to about 7.0.

In exemplary aspects, the methods comprise maintaining the cell culture at a temperature between 30° C. and 40° C. In exemplary embodiments, the temperature is between about 32° C. to about 38° C. or between about 35° C. to about 38° C.

In exemplary aspects, the methods comprise maintaining the osmolality between about 200 mOsm/kg to about 500 mOsm/kg. In exemplary aspects, the method comprises maintaining the osmolality between about 225 mOsm/kg to about 400 mOsm/kg or about 225 mOsm/kg to about 375 mOsm/kg. In exemplary aspects, the method comprises maintaining the osmolality between about 225 mOsm/kg to about 350 mOsm/kg. In various aspects, osmolality (mOsm/kg) is maintained at about 200, 225, about 250, about 275, about 300, about 325, about 350, about 375, about 400, about 425, about 450, about 475, or about 500.

In exemplary aspects, the methods comprise maintaining dissolved the oxygen (DO) level of the cell culture at about 20% to about 60% oxygen saturation during the initial cell culture period. In exemplary instances, the method comprises maintaining DO level of the cell culture at about 30% to about 50% (e.g., about 35% to about 45%) oxygen saturation during the initial cell culture period. In exemplary instances, the method comprises maintaining DO level of the cell culture at about 20%, about 25%, about 30%, about 35%, about 40%, about 45%, about 50%, about 55%, or about 60% oxygen saturation during the initial cell culture period. In exemplary aspects, the DO level is about 35 mm Hg to about 85 mmHg or about 40 mm Hg to about 80 mmHg or about 45 mm Hg to about 75 mm Hg.

The cell culture is maintained in any one or more culture medium. In exemplary aspects, the cell culture is maintained in a medium suitable for cell growth and/or is provided with one or more feeding media according to any suitable feeding schedule. In exemplary aspects, the method comprises maintaining the cell culture in a medium comprising glucose, lactate, ammonia, glutamine, and/or glutamate. In exemplary aspects, the method comprises maintaining the cell culture in a medium comprising manganese at a concentration less than or about 1 μM during the initial cell culture period. In exemplary aspects, the method comprises maintaining the cell culture in a medium comprising about 0.25 μM to about 1 μM manganese. In exemplary aspects, the method comprises maintaining the cell culture in a medium comprising negligible amounts of manganese. In exemplary aspects, the method comprises maintaining the cell culture in a medium comprising copper at a concentration less than or about 50 ppb during the initial cell culture period. In exemplary aspects, the method comprises maintaining the cell culture in a medium comprising copper at a concentration less than or about 40 ppb during the initial cell culture period. In exemplary aspects, the method comprises maintaining the cell culture in a medium comprising copper at a concentration less than or about 30 ppb during the initial cell culture period. In exemplary aspects, the method comprises maintaining the cell culture in a medium comprising copper at a concentration less than or about 20 ppb during the initial cell culture period. In exemplary aspects, the medium comprises copper at a concentration greater than or about 5 ppb or greater than or about 10 ppb. In exemplary aspects, the cell culture medium comprises mannose. In exemplary aspects, the cell culture medium does not comprise mannose.

In exemplary embodiments, the type of cell culture is a fed-batch culture or a continuous perfusion culture. However, the methods of the disclosure are advantageously not limited to any particular type of cell culture.

### Cells

The disclosure relates to methods of producing a recombinant glycosylated protein composition (e.g., an antibody composition or antibody binding protein composition) comprising maintaining glycosylation-competent cells in a cell culture medium. In exemplary aspects, the glycosylation-competent cells are eukaryotic cells, including, but not limited to, yeast cells, filamentous fungi cells, protozoa cells, algae cells, insect cells, or mammalian cells. Such host cells are described in the art. See, e.g., Frenzel, et al., Front Immunol 4:217 (2013). In exemplary aspects, the eukaryotic cells are mammalian cells. In exemplary aspects, the mammalian cells are non-human mammalian cells. In some aspects, the cells are Chinese Hamster Ovary (CHO) cells and derivatives thereof (e.g., CHO-K1, CHO pro-3), mouse myeloma cells (e.g., NS0, GS-NS0, Sp2/0), cells engineered to be deficient in dihydrofolatereductase (DHFR) activity (e.g., DUKX-X11, DG44), human embryonic kidney 293 (HEK293) cells or derivatives thereof (e.g., HEK293T, HEK293-EBNA), green African monkey kidney cells (e.g., COS cells, VERO cells), human cervical cancer cells (e.g., HeLa), human bone osteosarcoma epithelial cells U2-OS, adenocarcinoma human alveolar basal epithelial cells A549, human fibrosarcoma cells HT1080, mouse brain tumor cells CAD, embryonic carcinoma cells P19, mouse embryo fibroblast cells NIH 3T3, mouse fibroblast cells L929, mouse neuroblastoma cells N2a, human breast cancer cells MCF-7, retinoblastoma cells Y79, human retinoblastoma cells SO-Rb50, human liver cancer cells Hep G2, mouse B myeloma cells J558L, or baby hamster kidney (BHK) cells (Gaillet et al. 2007; Khan, Adv Pharm Bull 3(2): 257-263 (2013)).

Cells that are not glycosylation-competent can also be transformed into glycosylation-competent cells, e.g. by transfecting them with genes encoding relevant enzymes necessary for glycosylation. Exemplary enzymes include but are not limited to oligosaccharyltransferases, glycosidases, glucosidase I, glucosidease II, calnexin/calreticulin, glycosyltransferases, mannosidases, GlcNAc transferases, galactosyltransferases, and sialyltransferases.

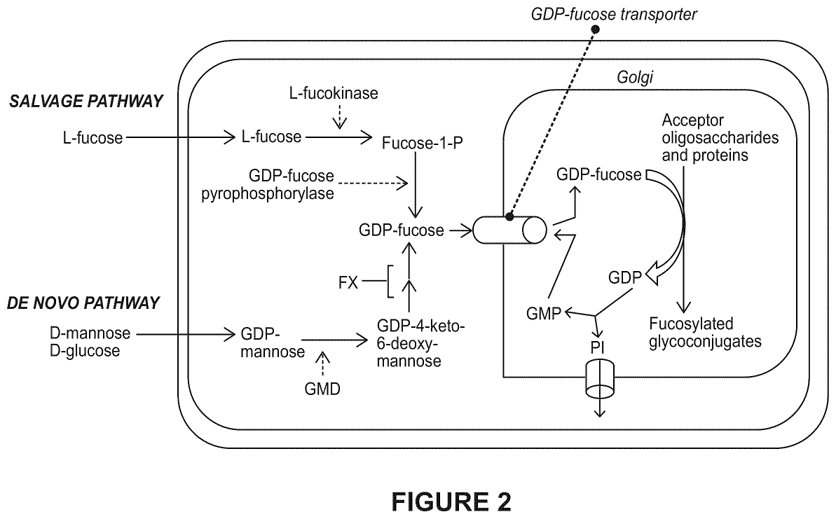

In exemplary embodiments, the glycosylation-competent cells are not genetically modified to alter the activity of an enzyme of the de novo pathway or the salvage pathway. These two pathways of fucose metabolism are shown in FIG. 2. In exemplary embodiments, the glycosylation-competent cells are not genetically modified to alter the activity of any one or more of: a fucosyl-transferase (FUT, e.g.,FUT1, FUT2, FUT3, FUT4, FUT5, FUT6, FUT7, FUT8, FUT9), a fucose kinase, a GDP-fucose pyrophosphorylase, GDP-D-mannose-4,6-dehydratase (GMD), and GDP-keto-6-deoxymannose-3,5-epimerase, 4-reductase (FX). In exemplary embodiments, the glycosylation-competent cells are not genetically modified to knock-out a gene encoding FX.

In exemplary embodiments, the glycosylation-competent cells are not genetically modified to alter the activity β(1,4)-N-acetylglucosaminyltransferase III (GNTIII) or GDP-6-deoxy-D-lyxo-4-hexulose reductase (RMD). In exemplary aspects, the glycosylation-competent cells are not genetically modified to overexpress GNTIII or RMD.

### Recombinant Glycosylated Proteins

In exemplary embodiments, the recombinant glycosylated protein comprises an amino acid sequence comprising one or more N-glycosylation consensus sequences of the formula:

Asn-Xaa₁-Xaa₂

wherein Xaa₁ is any amino acid except Pro, and Xaa₂ is Ser or Thr.

In exemplary embodiments, the recombinant glycosylated protein comprises a fragment crystallizable (Fc) polypeptide. The term “Fc polypeptide” as used herein includes native and mutein forms of polypeptides derived from the Fc region of an antibody. Truncated forms of such polypeptides containing the hinge region that promotes dimerization also are included. Fusion proteins comprising Fc moieties (and oligomers formed therefrom) offer the advantage of facile purification by affinity chromatography over Protein A or Protein G columns. In exemplary embodiments, the recombinant glycosylated protein comprises the Fc of an IgG, e.g., a human IgG. In exemplary aspects, the recombinant glycosylated protein comprises the Fc an IgG1 or IgG2. In exemplary aspects, the recombinant glycosylated protein is an antibody, an antibody protein product, a peptibody, or a Fc-fusion protein.

In exemplary aspects, the recombinant glycosylated protein is an antibody. As used herein, the term “antibody” refers to a protein having a conventional immunoglobulin format, comprising heavy and light chains, and comprising variable and constant regions. For example, an antibody may be an IgG which is a “Y-shaped” structure of two identical pairs of polypeptide chains, each pair having one “light” (typically having a molecular weight of about 25 kDa) and one “heavy” chain (typically having a molecular weight of about 50-70 kDa). An antibody has a variable region and a constant region. In IgG formats, the variable region is generally about 100-110 or more amino acids, comprises three complementarity determining regions (CDRs), is primarily responsible for antigen recognition, and substantially varies among other antibodies that bind to different antigens. See, e.g., Janeway et al., “Structure of the Antibody Molecule and the Immunoglobulin Genes”, Immunobiology: The Immune System in Health and Disease, 4^{th} ed. Elsevier Science Ltd./Garland Publishing, (1999).

Briefly, in an antibody scaffold, the CDRs are embedded within a framework in the heavy and light chain variable region where they constitute the regions largely responsible for antigen binding and recognition. A variable region comprises at least three heavy or light chain CDRs (Kabat et al., 1991, Sequences of Proteins of Immunological Interest, Public Health Service N.I.H., Bethesda, Md.; see also Chothia and Lesk, 1987, J. Mol. Biol. 196:901-917; Chothia et al., 1989, Nature 342: 877-883), within a framework region (designated framework regions 1-4, FR1, FR2, FR3, and FR4, by Kabat et al., 1991; see also Chothia and Lesk, 1987, supra).

Human light chains are classified as kappa and lambda light chains. Heavy chains are classified as mu, delta, gamma, alpha, or epsilon, and define the antibody's isotype as IgM, IgD, IgG, IgA, and IgE, respectively. IgG has several subclasses, including, but not limited to IgG1, IgG2, IgG3, and IgG4. IgM has subclasses, including, but not limited to, IgM1 and IgM2. Embodiments of the disclosure include all such classes or isotypes of antibodies. The light chain constant region can be, for example, a kappa- or lambda-type light chain constant region, e.g., a human kappa- or lambda-type light chain constant region. The heavy chain constant region can be, for example, an alpha-, delta-, epsilon-, gamma-, or mu-type heavy chain constant regions, e.g., a human alpha-, delta-, epsilon-, gamma-, or mu-type heavy chain constant region. Accordingly, in exemplary embodiments, the antibody is an antibody of isotype IgA, IgD, IgE, IgG, or IgM, including any one of IgG1, IgG2, IgG3 or IgG4.

In various aspects, the antibody can be a monoclonal antibody or a polyclonal antibody. In some aspects, the antibody comprises a sequence that is substantially similar to a naturally-occurring antibody produced by a mammal, e.g., mouse, rat, rabbit, goat, horse, chicken, hamster, pig, human, and the like. In this regard, the antibody may be considered as a mammalian antibody, e.g., a mouse antibody, rat antibody, rabbit antibody, goat antibody, horse antibody, chicken antibody, hamster antibody, pig antibody, human antibody, and the like. In certain aspects, the recombinant glycosylated protein is a monoclonal human antibody. In certain aspects, the recombinant glycosylated protein is a chimeric antibody or a humanized antibody. The term “chimeric antibody” is used herein to refer to an antibody containing constant domains from one species and the variable domains from a second, or more generally, containing stretches of amino acid sequence from at least two species. The term “humanized” when used in relation to antibodies refers to antibodies having at least CDR regions from a non-human source which are engineered to have a structure and immunological function more similar to true human antibodies than the original source antibodies. For example, humanizing can involve grafting CDR from a non-human antibody, such as a mouse antibody, into a human antibody. Humanizing also can involve select amino acid substitutions to make a non-human sequence look more like a human sequence.

An antibody, in various aspects, is cleaved into fragments by enzymes, such as, e.g., papain and pepsin. Papain cleaves an antibody to produce two Fab fragments and a single Fc fragment. Pepsin cleaves an antibody to produce a F(ab′)₂ fragment and a pFc′ fragment. In exemplary aspects, the recombinant glycosylated protein is an antibody fragment, e.g., a Fab, Fc, F(ab′)₂, or a pFc′, that retains at least one glycosylation site. With regard to the methods of the disclosure, the antibody may lack certain portions of an antibody, and may be an antibody fragment. In various aspects, the antibody fragment comprises a glycosylation site. In some aspects, the fragment is a “Glycosylated Fc Fragment” which comprises at least a portion of the Fc region of an antibody which is glycosylated post-translationally in eukaryotic cells.

The architecture of antibodies has been exploited to create a growing range of alternative antibody formats that spans a molecular-weight range of at least or about 12-150 kDa and a valency (n) range from monomeric (n=1), dimeric (n=2) and trimeric (n=3) to tetrameric (n=4) and potentially higher; such alternative antibody formats are referred to herein as “antibody protein products” or “antibody binding proteins”.

Antibody protein products can be an antigen binding format based on antibody fragments, e.g., scFvs, Fabs and VHH/VH, which retain full antigen-binding capacity. The smallest antigen-binding fragment that retains its complete antigen binding site is the Fv fragment, which consists entirely of variable (V) regions. A soluble, flexible amino acid peptide linker is used to connect the V regions to a scFv (single chain fragment variable) fragment for stabilization of the molecule, or the constant (C) domains are added to the V regions to generate a Fab fragment [fragment, antigen-binding]. Both scFv and Fab are widely used fragments that can be easily produced in prokaryotic hosts. Other antibody protein products include disulfide-bond stabilized scFv (ds-scFv), single chain Fab (scFab), as well as di- and multimeric antibody formats like dia-, tria- and tetra-bodies, or minibodies (miniAbs) that comprise different formats consisting of scFvs linked to oligomerization domains. The smallest fragments are VHH/VH of camelid heavy chain Abs as well as single domain Abs (sdAb). The building block that is most frequently used to create novel antibody formats is the single-chain variable (V)-domain antibody fragment (scFv), which comprises V domains from the heavy and light chain (VH and VL domain) linked by a peptide linker of ˜15 amino acid residues. A peptibody or peptide-Fc fusion is yet another antibody protein product. The structure of a peptibody consists of a biologically active peptide grafted onto an Fc domain. Peptibodies are well-described in the art. See, e.g., Shimamoto et al., mAbs 4(5): 586-591 (2012).

Other antibody protein products include a single chain antibody (SCA); a diabody; a triabody; a tetrabody; bispecific or trispecific antibodies, and the like. Bispecific antibodies can be divided into five major classes: BsIgG, appended IgG, BsAb fragments, bispecific fusion proteins and BsAb conjugates. See, e.g., Spiess et al., Molecular Immunology 67(2) Part A: 97-106 (2015).

In exemplary aspects, the recombinant glycosylated protein comprises any one of these antibody protein products (e.g., scFv, Fab VHH/VH, Fv fragment, ds-scFv, scFab, dimeric antibody, multimeric antibody (e.g., a diabody, triabody, tetrabody), miniAb, peptibody VHH/VH of camelid heavy chain antibody, sdAb, diabody; a triabody; a tetrabody; a bispecific or trispecific antibody, BsIgG, appended IgG, BsAb fragment, bispecific fusion protein, and BsAb conjugate) and comprises one or more N-glycosylation consensus sequences, optionally, one or more Fc polypeptides. In various aspects, the antibody protein product comprises a glycosylation site. In exemplary aspects, an antibody protein product can be a Glycosylated Fc Fragment conjugated to an antibody binding fragment (“Glycosylated Fc Fragment antibody product”).

The recombinant glycosylated protein may be an antibody protein product in monomeric form, or polymeric, oligomeric, or multimeric form. In certain embodiments in which the antibody comprises two or more distinct antigen binding regions fragments, the antibody is considered bispecific, trispecific, or multi-specific, or bivalent, trivalent, or multivalent, depending on the number of distinct epitopes that are recognized and bound by the antibody.

Advantageously, the methods are not limited to the antigen-specificity of the antibody. Accordingly, the antibody has any binding specificity for virtually any antigen. In exemplary aspects, the antibody binds to a hormone, growth factor, cytokine, a cell-surface receptor, or any ligand thereof. In exemplary aspects, the antibody binds to a protein expressed on the cell surface of an immune cell. In exemplary aspects, the antibody binds to a cluster of differentiation molecule selected from the group consisting of: CD1a, CD1b, CD1c, CD1d, CD2, CD3, CD4, CD5, CD6, CD7, CD8, CD9, CD10, CD11A, CD11B, CD11C, CDw12, CD13, CD14, CD15, CD15s, CD16, CDw17, CD18, CD19, CD20, CD21, CD22, CD23, CD24, CD25, CD26, CD27, CD28, CD29, CD30, CD31, CD32, CD33, CD34, CD35, CD36, CD37, CD38, CD39, CD40, CD41, CD42a, CD42b, CD42c, CD42d, CD43, CD44, CD45, CD45RO, CD45RA, CD45RB, CD46, CD47, CD48, CD49a, CD49b, CD49c, CD49d, CD49e, CD49f, CD50, CD51, CD52, CD53, CD54, CD55, CD56, CD57, CD58, CD59, CDw60, CD61, CD62E, CD62L, CD62P, CD63, CD64, CD65, CD66a, CD66b, CD66c, CD66d, CD66e, CD66f, CD68, CD69, CD70, CD71, CD72, CD73, CD74, CD75, CD76, CD79a, CD79B, CD80, CD81, CD82, CD83, CDw84, CD85, CD86, CD87, CD88, CD89, CD90, CD91, CDw92, CD93, CD94, CD95, CD96, CD97, CD98, CD99, CD100, CD101, CD102, CD103, CD104, CD105, CD106, CD107a, CD107b, CDw108, CD109, CD114, CD115, CD116, CD117, CD118, CD119, CD120a, CD120b, CD121a, CDw121b, CD122, CD123, CD124, CD125, CD126, CD127, CDw128, CD129, CD130, CDw131, CD132, CD134, CD135, CDw136, CDw137, CD138, CD139, CD140a, CD140b, CD141, CD142, CD143, CD144, CD145, CD146, CD147, CD148, CD150, CD151, CD152, CD153, CD154, CD155, CD156, CD157, CD158a, CD158b, CD161, CD162, CD163, CD164, CD165, CD166, and CD182.

In exemplary aspects, the antibody is one of those described in U.S. Pat. No. 7,947,809 and U.S. Patent Application Publication No. 20090041784 (glucagon receptor), U.S. Pat. Nos. 7,939,070, 7,833,527, 7,767,206, and 7,786,284 (IL-17 receptor A), U.S. Pat. Nos. 7,872,106 and 7,592,429 (Sclerostin), U.S. Pat. Nos. 7,871,611, 7,815,907, 7,037,498, 7,700,742, and U.S. Patent Application Publication No. 20100255538 (IGF-1 receptor), U.S. Pat. No. 7,868,140 (B7RP1), U.S. Pat. No. 7,807,159 and U.S. Patent Application Publication No. 20110091455 (myostatin), U.S. Pat. No. 7,736,644, 7,628,986, 7,524,496, and U.S. Patent Application Publication No. 20100111979 (deletion mutants of epidermal growth factor receptor), U.S. Pat. No. 7,728,110 (SARS coronavirus), U.S. Pat. No. 7,718,776 and U.S. Patent Application Publication No. 20100209435 (OPGL), U.S. Pat. Nos. 7,658,924 and 7,521,053 (Angiopoietin-2), U.S. Pat. Nos. 7,601,818, 7,795,413, U.S. Patent Application Publication No. 20090155274, U.S. Patent Application Publication No. 20110040076 (NGF), U.S. Pat. No. 7,579,186 (TGF-type II receptor), U.S. Pat. No. 7,541,438 (connective tissue growth factor), U.S. Pat. No. 7,438,910 (IL1-R1), U.S. Pat. No. 7,423,128 (properdin), U.S. Pat. No. 7,411,057, 7,824,679, 7,109,003, 6,682,736, 7,132,281, and 7,807,797 (CTLA-4), U.S. Pat. No. 7,084,257, 7,790,859, 7,335,743, 7,084,257, and U.S. Patent Application Publication No. 20110045537 (interferon-gamma), U.S. Pat. No. 7,932,372 (MAdCAM), U.S. Pat. No. 7,906,625, U.S. Patent Application Publication No. 20080292639, and U.S. Patent Application Publicaiton No. 20110044986 (amyloid), U.S. Pat. Nos. 7,815,907 and 7,700,742 (insulin-like growth factor I), U.S. Pat. Nos. 7,566,772 and 7,964,193 (interleukin-1B), U.S. Pat. Nos. 7,563,442, 7,288,251, 7,338,660, 7,626,012, 7,618,633, and U.S. Patent Application Publication No. 20100098694 (CD40), U.S. Pat. No. 7,498,420 (c-Met), U.S. Pat. Nos. 7,326,414, 7,592,430, and 7,728,113 (M-CSF), U.S. Pat. No. 6,924,360, 7,067,131, and 7,090,844 (MUC18), U.S. Pat. Nos. 6,235,883, 7,807,798, and U.S. Patent Application Publication No. 20100305307 (epidermal growth factor receptor), U.S. Pat. Nos. 6,716,587, 7,872,113, 7,465,450, 7,186,809, 7,317,090, and 7,638,606 (interleukin-4 receptor), U.S. Patent Application Publication No. 20110135657 (BETA-KLOTHO), U.S. Pat. Nos. 7,887,799 and 7,879,323 (fibroblast growth factor-like polypeptides), U.S. Pat. No. 7,867,494 (IgE), U.S. Patent Application Publication No. 20100254975 (ALPHA-4 BETA-7), U.S. Patent Application Publication No. 20100197005 and U.S. Pat. No. 7,537,762 (ACTIVIN RECEPTOR-LIKE KINASE-1), U.S. Pat. No. 7,585,500 and U.S. Patent Application Publication No. 20100047253 (IL-13), U.S. Patent Application Publication No. 20090263383 and U.S. Pat. No. 7,449,555 (CD148), U.S. Patent Application Publication No. 20090234106 (ACTIVIN A), U.S. Patent Application Publication No. 20090226447 (angiopoietin-1 and angiopoietin-2), U.S. Patent Application Publication No. 20090191212 (Angiopoietin-2), U.S. Patent Application Publicaiton No. 20090155164 (C-FMS), U.S. Pat. No. 7,537,762 (activin receptor-like kinase-1), U.S. Pat. No. 7,371,381 (galanin), U.S. Patent Application Publication No. 20070196376 (INSULIN-LIKE GROWTH FACTORS), U.S. Pat. Nos. 7,267,960 and 7,741,115 (LDCAM), U.S. Pat. No. 7,265,212 (CD45RB), U.S. Pat. No. 7,709,611, U.S. Patent Application Publication No. 20060127393 and U.S. Patent Application Publication No. 20100040619 (DKK1), U.S. Pat. No. 7,807,795, U.S. Patent Application Publication No. 20030103978 and U.S. Pat. No. 7,923,008 (osteoprotegerin), U.S. Patent Application Publication No. 20090208489 (OV064), U.S. Patent Application Publication No. 20080286284 (PSMA), U.S. Pat. No. 7,888,482, U.S. Patent Application Publication No. 20110165171, and U.S. Patent Application Publication No. 20110059063 (PAR2), U.S. Patent Application Publication No. 20110150888 (HEPCIDIN), U.S. Pat. No. 7,939,640 (B7L-1), U.S. Pat. No. 7,915,391 (c-Kit), U.S. Pat. Nos. 7,807,796, 7,193,058, and U.S. Pat. No. 7,427,669 (ULBP), U.S. Pat. Nos. 7,786,271, 7,304,144, and U.S. Patent Application Publication No. 20090238823 (TSLP), U.S. Pat. No. 7,767,793 (SIGIRR), U.S. Pat. No. 7,705,130 (HER-3), U.S. Pat. No. 7,704,501 (ataxin-1-like polypeptide), U.S. Pat. Nos. 7,695,948 and 7,199,224 (TNF-α converting enzyme), U.S. Patent Application Publication No. 20090234106 (ACTIVIN A), U.S. Patent Application Publication No. 20090214559 and U.S. Pat. No. 7,438,910 (IL1-R1), U.S. Pat. No. 7,579,186 (TGF-type II receptor), U.S. Pat. No. 7,569,387 (TNF receptor-like molecules), U.S. Pat. No. 7,541,438, (connective tissue growth factor), U.S. Pat. No. 7,521,048 (TRAIL receptor-2), U.S. Pat. Nos. 6,319,499, 7,081,523, and U.S. Patent Application Publication No. 20080182976 (erythropoietin receptor), U.S. Patent Application Publication No. 20080166352 and U.S. Pat. No. 7,435,796 (B7RP1), U.S. Pat. No. 7,423,128 (properdin), U.S. Pat. Nos. 7,422,742 and 7,141,653 (interleukin-5), U.S. Pat. Nos. 6,740,522 and 7,411,050 (RANKL), U.S. Pat. No. 7,378,091 (carbonic anhydrase IX (CA IX) tumor antigen), U.S. Pat. Nos. 7,318,925 and 7,288,253 (parathyroid hormone), U.S. Pat. No. 7,285,269 (TNF), U.S. Pat. No. 6,692,740 and U.S. Pat. No. 7,270,817 (ACPL), U.S. Pat. No. 7,202,343 (monocyte chemo-attractant protein-1), U.S. Pat. No. 7,144,731 (SCF), U.S. Pat. Nos. 6,355,779 and 7,138,500 (4-1BB), U.S. Pat. No. 7,135,174 (PDGFD), U.S. Pat. Nos. 6,630,143 and 7,045,128 (Flt-3 ligand), U.S. Pat. No. 6,849,450 (metalloproteinase inhibitor), U.S. Pat. No. 6,596,852 (LERK-5), U.S. Pat. No. 6,232,447 (LERK-6), U.S. Pat. No. 6,500,429 (brain-derived neurotrophic factor), U.S. Pat. No. 6,184,359 (epithelium-derived T-cell factor), U.S. Pat. No. 6,143,874 (neurotrophic factor NNT-1), U.S. Patent Application Publication No. 20110027287 (PROPROTEIN CONVERTASE SUBTILISIN KEXIN TYPE 9 (PCSK9)), U.S. Patent Application Publication No. 20110014201 (IL-18 RECEPTOR), and U.S. Patent Application Publication No. 20090155164 (C-FMS). The above patents and published patent applications are incorporated herein by reference in their entirety for purposes of their disclosure of variable domain polypeptides, variable domain encoding nucleic acids, host cells, vectors, methods of making polypeptides encoding said variable domains, pharmaceutical compositions, and methods of treating diseases associated with the respective target of the variable domain-containing antigen binding protein or antibody.

In exemplary embodiments, the antibody is one of Muromonab-CD3 (product marketed with the brand name Orthoclone Okt3®), Abciximab (product marketed with the brand name Reopro®.), Rituximab (product marketed with the brand name MabThera®, Rituxan®), Basiliximab (product marketed with the brand name Simulect®), Daclizumab (product marketed with the brand name Zenapax®), Palivizumab (product marketed with the brand name Synagis®), Infliximab (product marketed with the brand name Remicade®), Trastuzumab (product marketed with the brand name Herceptin®), Alemtuzumab (product marketed with the brand name MabCampath®, Campath-1H®), Adalimumab (product marketed with the brand name Humira®), Tositumomab-I 131 (product marketed with the brand name Bexxar®), Efalizumab (product marketed with the brand name Raptiva®), Cetuximab (product marketed with the brand name Erbitux®), I'Ibritumomab tiuxetan (product marketed with the brand name Zevalin®), I'Omalizumab (product marketed with the brand name Xolair®), Bevacizumab (product marketed with the brand name Avastin®), Natalizumab (product marketed with the brand name Tysabri®), Ranibizumab (product marketed with the brand name Lucentis®), Panitumumab (product marketed with the brand name Vectibix®), I'Eculizumab (product marketed with the brand name Soliris®), Certolizumab pegol (product marketed with the brand name Cimzia®), Golimumab (product marketed with the brand name Simponi®), Canakinumab (product marketed with the brand name Ilaris®), Catumaxomab (product marketed with the brand name Removab®), Ustekinumab (product marketed with the brand name Stelara®), Tocilizumab (product marketed with the brand name RoActemra®, Actemra®), Ofatumumab (product marketed with the brand name Arzerra®), Denosumab (product marketed with the brand name Prolia®), Belimumab (product marketed with the brand name Benlysta®), Raxibacumab, Ipilimumab (product marketed with the brand name Yervoy®), and Pertuzumab (product marketed with the brand name Perjeta®). In exemplary embodiments, the antibody is one of anti-TNF alpha antibodies such as adalimumab, infliximab, etanercept, golimumab, and certolizumab pegol; anti-IL1.beta. antibodies such as canakinumab; anti-IL12/23 (p40) antibodies such as ustekinumab and briakinumab; and anti-IL2R antibodies, such as daclizumab. In exemplary aspects, the antibody binds to a tumor associated antigen and is an anti-cancer antibody. Examples of suitable anti-cancer antibodies include, but are not limited to, anti-BAFF antibodies such as belimumab; anti-CD20 antibodies such as rituximab; anti-CD22 antibodies such as epratuzumab; anti-CD25 antibodies such as daclizumab; anti-CD30 antibodies such as iratumumab, anti-CD33 antibodies such as gemtuzumab, anti-CD52 antibodies such as alemtuzumab; anti-CD152 antibodies such as ipilimumab; anti-EGFR antibodies such as cetuximab; anti-HER2 antibodies such as trastuzumab and pertuzumab; anti-IL6 antibodies, such as siltuximab; and anti-VEGF antibodies such as bevacizumab; anti-IL6 receptor antibodies such as tocilizumab. In exemplary aspects, the tumor associated antigen is CD20 and the antibody is an anti-CD20 antibody. In exemplary aspects, the tumor associated antigen comprises SEQ ID NO: 3. In exemplary instances, the antibody comprises an amino acid sequence of SEQ ID NO: 1 and an amino acid sequence of SEQ ID NO: 2. In exemplary aspects, the antibody is an anti-CD20 antibody, e.g., an anti-CD20 monoclonal antibody. In alternative aspects, the IgG1 antibody is rituximab, or a biosimilar thereof. The term rituximab refers to an IgG1 kappa chimeric murine/human, monoclonal antibody that binds CD20 antigen (see CAS Number: 174722-31-7; DrugBank-DB00073; Kyoto Encyclopedia of Genes and Genomes (KEGG) entry D02994). In exemplary aspects, the antibody comprises a light chain comprising a CDR1, CDR2, and CDR3 as set forth in Table A. In exemplary aspects, the antibody comprises a heavy chain comprising a CDR1, CDR2, and CDR3 as set forth in Table A. In various instances, the antibody comprises the VH and VL or comprising VH-IgG1 and VL-IgG kappa sequences recited in Table A.

TABLE A
Rituximab Amino Acid Sequences

| | | SEQ ID |
|---|---|---|
| Description | Sequence | NO: |
| LC CDR1 | RASSSVSYIH | 4 |
| LC CDR2 | ATSNLAS | 5 |
| LC CDR3 | QQWTSNPPT | 6 |
| HC CDR1 | SYNMH | 7 |
| HC CDR2 | AIYPGNGDTSYNQKFKG | 8 |
| HC CDR3 | STYYGGDWYFNV | 9 |
| VL | QIVLSQSPAILSASPGEKVTMTC | 10 |
| | RASSSVSYIHWFQQKPGSSPKPW | |
| | IYATSNLASGVPVRFSGSGSGTS | |
| | YSLTISRVEAEDAATYYCQQWTS | |
| | NPPTFQGGTKLEIK | |
| VH | QVQLQQPGAELVKPGASVKMSCK | 11 |
| | ASGYTFTSYNMHWVKQTPGRGLE | |
| | WIGAIYPGNGDTSYNQKFKGKAT | |
| | LTADKSSSTAYMQLSSLTSEDSA | |
| | VYYCARSTYYGGDWYFNVWGAGT | |
| | TVTVSA | |
| VL-IgG | QIVLSQSPAILSASPGEKVTMTC | 12 |
| Kappa | RASSSVSYIHWFQQKPGSSPKPW | |
| | IYATSNLASGVPVRFSGSGSGTS | |
| | YSLTISRVEAEDAATYYCQQWTS | |
| | NPPTFGGGTKLEIKRTVAAPSVF | |
| | IFPPSDEQLKSGTASVVCLLNNF | |
| | YPREAKVQWKVDNALQSGNSQES | |
| | VTEQDSKDSTYSLSSTLTLSKAD | |
| | YEKHKVYACEVTHQGLSSPVTKS | |
| | FNRGEC | |
| VH-IgG1 | QVQLQQPGAELVKPGASVKMSCK | 13 |
| | ASGYTFTSYNMHWVKQTPGRGLE | |
| | WIGAIYPGNGDTSYNQKFKGKAT | |
| | LTADKSSSTAYMQLSSLTSEDSA | |
| | VYYCARSTYYGGDWYFNVWGAGT | |
| | TVTVSAASTKGPSVFPLAPSSKS | |
| | TSGGTAALGCLVKDYFPEPVTVS | |
| | WNSGALTSGVHTFPAVLQSSGLY | |
| | SLSSVVTVPSSSLGTQTYICNVN | |
| | HKPSNTKVDKKAEPKSCDKTHTC | |
| | PPCPAPELLGGPSVFLFPPKPKD | |
| | TLMISRTPEVTCVVVDVSHEDPE | |
| | VKFNWYVDGVEVHNAKTKPREEQ | |
| | YNSTYRVVSVLTVLHQDWLNGKE | |
| | YKCKVSNKALPAPIEKTISKAKG | |
| | QPREPQVYTLPPSRDELTKNQVS | |
| | LTCLVKGFYPSDIAVEWESNGQP | |
| | ENNYKTTPPVLDSDGSFFLYSKL | |
| | TVDKSRWQGGNVFSCSVMHEALH | |
| | NHYTQKSLSLSPGK | |

LC, light chain;
HC, heavy chain;
VL, variable light chain;
VH, variable heavy chain.

In exemplary aspects, the antibody is an anti-EGFR antibody, e.g., an anti-HER2 monoclonal antibody. In exemplary aspects, the antibody is trastuzumab, or a biosimilar thereof. The term trastuzumab refers to an IgG1 kappa humanized, monoclonal antibody that binds HER2/neu antigen (see CAS Number: 180288-69-1; DrugBank-DB00072; Kyoto Encyclopedia of Genes and Genomes (KEGG) entry D03257). In exemplary aspects, the antibody comprises a light chain comprising a CDR1, CDR2, and CDR3 as set forth in Table B. In exemplary aspects, the antibody comprises a heavy chain comprising a CDR1, CDR2, and CDR3 as set forth in Table B. In various instances, the antibody comprises the VH and VL or comprising VH-IgG1 and VL-IgG kappa sequences recited in Table B.

TABLE B
Trastuzumab Amino Acid Sequences

| Description | Sequence | SEQ ID NO: |
|---|---|---|
| LC CDR1 | QDVNTA | 14 |
| LC CDR2 | SAS | 15 |
| LC CDR3 | QQHYTTPPT | 16 |
| HC CDR1 | GFNIKDTY | 17 |
| HC CDR2 | IYPTNGYT | 18 |
| HC CDR3 | SRWGGDGFYAMDY | 19 |
| VL | DIQMTQSPSSLSASVGDRVTITC | 20 |
| | RASQDVNTAVAWYQQKPGKAPKL | |
| | LIYSASFLYSGVPSRFSGSRSGT | |
| | DFTLTISSLQPEDFATYYCQQHY | |
| | TTPPTFGQGTKVEIK | |
| VH | EVQLVESGGGLVGPGGSLRLSCA | 21 |
| | ASGFNIKDTYIHWVRQAPGKGLE | |
| | WVARIYPTNGYTRYADSVKGRFT | |
| | ISADTSKNTAYLQMNSLRAEDTA | |
| | VYYCSRWGGDGFYAMDYWGQGTL | |
| | VTVSS | |
| VL-IgG | DIQMTQSPSSLSASVGDRVTITC | 22 |
| Kappa | RASQDVNTAVAWYQQKPGKAPKL | |
| | LIYSASFLYSGVPSRFSGSRSGT | |
| | DFTLTISSLQPEDFATYYCQQHY | |
| | TTPPTFGQGTKVEIKRTVAAPSV | |
| | FIFPPSDEQLKSGTASVVCLLNN | |
| | FYPREAKVQWKVDNALQSGNSQE | |
| | SVTEQDSKDSTYSLSSTLTLSKA | |
| | DYEKHKVYACEVTHQGLSSPVTK | |
| | SFNRGEC | |
| VH-IgG1 | EVQLVESGGGLVGPGGSLRLSCA | 23 |
| | ASGFNIKDTYIHWVRGAPGKGLE | |
| | WVARIYPTNGYTRYADSVKGRFT | |
| | ISADTSKNTAYLQMNSLRAEDTA | |
| | VYYCSRVVGGDGFYAMDYWGQGT | |
| | LVTVSSASTKGPSVFPLAPSSKS | |
| | TSGGTAALGCLVKDYFPEPVTVS | |
| | WNSGALTSGVHTFPAVLQSSGLY | |
| | SLSSVVTVPSSSLGTQTYICNVN | |
| | HKPSNTKVDKKVEPPKSCDKTHT | |
| | CPPCPAPELLGGPSVFLFPPKPK | |
| | DTLMISRTPEVTCVVVDVSHEDP | |
| | EVKFNWYVDGVEVHNAKTKPREE | |
| | QYNSTYRVVSVLTVLHQDWLNGK | |
| | EYKCKVSNKALPAPIEKTISKAK | |
| | GQPREPQVYTLPPSRDELTKNQV | |
| | SLTCLVKGFYPSDIAVEWESNGQ | |
| | PENNYKTTPPVLDSDGSFFLYSK | |
| | LTVDKSRWQQGNVFSCSVMHEAL | |
| | HNHYTQKSLSLSPG | |

LC, light chain;
HC, heavy chain;
VL, variable light chain;
VH, variable heavy chain.

### Additional Steps

The methods disclosed herein, in various aspects, comprise additional steps. For example, in some aspects, the methods comprise one or more upstream steps or downstream steps involved in producing, purifying, and formulating a recombinant glycosylated protein. In exemplary embodiments, the method comprises steps for generating host cells that express a recombinant glycosylated protein (e.g., antibody or antibody binding protein). The host cells, in some aspects, are prokaryotic host cells, e.g., E. coli or Bacillus subtilis, or the host cells, in some aspects, are eukaryotic host cells, e.g., yeast cells, filamentous fungi cells, protozoa cells, insect cells, or mammalian cells (e.g., CHO cells). Such host cells are described in the art. See, e.g., Frenzel, et al., Front Immunol 4:217 (2013) and herein under “Cells.” For example, the methods comprise, in some instances, introducing into host cells a vector comprising a nucleic acid comprising a nucleotide sequence encoding the recombinant glycosylated protein, or a polypeptide chain thereof.

In exemplary embodiments, the methods disclosed herein comprise steps for isolating and/or purifying the recombinant glycosylated protein (e.g., recombinant antibody) from the culture. In exemplary aspects, the method comprises one or more chromatography steps including, but not limited to, e.g., affinity chromatography (e.g., protein A affinity chromatography), ion exchange chromatography, and/or hydrophobic interaction chromatography. In exemplary aspects, the method comprises steps for producing crystalline biomolecules from a solution comprising the recombinant glycosylated proteins.

The methods of the disclosure, in various aspects, comprise one or more steps for preparing a composition, including, in some aspects, a pharmaceutical composition, comprising the purified recombinant glycosylated protein. Such compositions are discussed below.

### Compositions

Provided herein are compositions comprising recombinant glycosylated proteins. In exemplary embodiments, the compositions are prepared by the inventive methods of producing a recombinant glycosylated protein composition, described herein. In exemplary aspects, the recombinant glycosylated protein is an antibody. Accordingly, antibody compositions are provided herein. In exemplary embodiments, the antibody composition comprises different glycoforms of the antibody. In exemplary embodiments, the antibody composition comprises TAF glycoforms, HM glycoforms, and/or afucosylated glycoforms of the antibody. Compositions comprising antibody fragments or antibody protein products are also provided. In various aspects, the antibody fragments, antibody protein products, Glycosylated Fc Fragments, or Glycosylated Fc Fragment antibody products comprise a glycosylation site. In exemplary embodiments, the antibody composition is produced by a method comprising maintaining glycosylation-competent cells in a cell culture medium comprising fucose, wherein fucose is present in the culture medium at a concentration between about 0.17 g/L and about 1.0 g/L. In exemplary embodiments, the antibody composition is produced by a method comprising maintaining glycosylation-competent cells in a cell culture medium comprising fucose, wherein fucose is present in the culture medium at a concentration between about 0.1 g/L and about 1.0 g/L, and wherein the glycosylation-competent cells are not genetically modified to alter activity of an enzyme of the de novo pathway or the salvage pathway. In exemplary embodiments, the antibody composition is produced by a method comprising maintaining glycosylation-competent cells in a cell culture medium comprising fucose and glucose, wherein fucose is present in the culture medium at a concentration of about 0.1 g/L to about 1.0 g/L and adding glucose to the cell culture medium according to a glucose feeding schedule that achieves an average glucose concentration of about 10 g/L or less. In exemplary embodiments, the antibody composition is produced upon practicing a method of modulating the level of TAF glycans, afucosylated glycans, or high mannose glycan of an antibody composition produced by glycosylation-competent cells. In exemplary aspects, the antibody composition is produced upon practicing a method of modulating the level of TAF glycans comprising (A) adding fucose to a cell culture medium comprising the glycosylation-competent cells to achieve a fucose concentration of about 0.1 g/L to about 1.0 g/L to decrease the level of TAF glycans; (B) adding glucose to a cell culture medium comprising the glycosylation-competent cells to achieve a glucose concentration less than about 10 g/L to increase the level of TAF glycans; or (C) both (A) and (B). In exemplary aspects, the antibody composition is produced upon practicing a method of modulating the level of afucosylated glycans of an antibody composition produced by glycosylation-competent cells, comprising (A) adding fucose to a cell culture medium comprising the glycosylation-competent cells to achieve a fucose concentration of about 0.1 g/L to about 1.0 g/L to decrease the level of afucosylated glycans; (B) adding glucose to a cell culture medium comprising the glycosylation-competent cells to achieve a glucose concentration less than or about 10 g/L to increase the level of afucosylated glycans; or (C) both (A) and (B). In exemplary instances, the antibody composition is produced upon practicing a method of modulating the level of high mannose glycans of an antibody composition produced by glycosylation-competent cells, comprising adding glucose to a cell culture medium comprising the glycosylation-competent cells to achieve a glucose concentration less than or about 10 g/L to increase the level of high mannose glycans.

In exemplary aspects, less than or about 50% (e.g., less than or about 40%, less than or about 30%, less than or about 25%, less than or about 20%, less than or about 15%) of the recombinant glycosylated protein in the composition are TAF glycoforms. In exemplary aspects, less than about 10% (e.g., less than or about 9%, less than or about 8%, less than or about 7%, less than or about 6%, less than or about 5%, less than or about 4%, less than or about 3%, less than or about 2%) of the recombinant glycosylated protein in the composition are TAF glycoforms. In exemplary aspects, about 4% to about 10% of the recombinant glycosylated protein in the composition are TAF glycoforms. In exemplary aspects, about 2% to about 6% of the recombinant glycosylated protein in the composition are TAF glycoforms. In exemplary aspects, about 2.5% to about 5% of the recombinant glycosylated protein in the composition are TAF glycoforms. In exemplary aspects, less than or about 4% of the recombinant glycosylated protein in the composition are TAF glycoforms. In further exemplary aspects, less than or about 4% and greater than or about 2% of the recombinant glycosylated protein in the composition are TAF glycoforms.

In exemplary aspects, the compositions of the disclosure have a glycoform profile which is less than or about 50% (e.g., less than or about 40%, less than or about 30%, less than or about 25%, less than or about 20%, less than or about 15%) TAF glycoforms. In exemplary aspects, the compositions of the disclosure have a glycoform profile which is less than or about 10% (e.g., less than or about 9%, less than or about 8%, less than or about 7%, less than or about 6%, less than or about 5%, less than or about 4%, less than or about 3%, less than or about 2%) TAF glycoforms. In exemplary aspects, the compositions of the disclosure have a glycoform profile which comprises about 4% to about 10% TAF glycoforms. In exemplary aspects, the compositions of the disclosure have a glycoform profile which is about 2% to about 6% TAF glycoforms. In exemplary aspects, the compositions of the disclosure have a glycoform profile which is about 2.5% to about 5% TAF glycoforms. In exemplary aspects, the compositions of the disclosure have a glycoform profile which is less than or about 4% TAF glycoforms. In exemplary aspects, the compositions of the disclosure have a glycoform profile which is less than or about 4% and greater than or about 2% TAF glycoforms.

In exemplary aspects, less than or about 5% of the recombinant glycosylated protein (e.g., antibody or antibody binding protein) in the composition are afucosylated glycoforms. In exemplary aspects, less than or about 4% of the recombinant glycosylated protein (e.g., antibody or antibody binding protein) in the composition are afucosylated glycoforms. In exemplary aspects, less than or about 3.5% of the recombinant glycosylated protein (e.g., antibody or antibody binding protein) in the composition are afucosylated glycoforms. In exemplary aspects, about 0.8% to about 2.8% of the recombinant glycosylated protein in the composition are afucosylated glycoforms. In some aspects, the level of afucosylated glycans in the antibody composition is about 0.8%, about 0.9%, about 1.0%, about 1.1%, about 1.2%, about 1.3%, about 1.4%, about 1.5%, about 1.6%, about 1.7%, about 1.8%, about 1.9%, about 2.0%, about 2.1%, about 2.2%, about 2.3%, about 2.4%, about 2.5%, about 2.6%, about 2.7%, or about 2.8%.

In exemplary aspects, less than or about 5% of the recombinant glycosylated protein (e.g., antibody or antibody binding protein) in the composition are high mannose glycoforms. In exemplary aspects, less than or about 4% of the recombinant glycosylated protein (e.g., antibody or antibody binding protein) in the composition are high mannose glycoforms. In exemplary aspects, less than or about 3.5% of the recombinant glycosylated protein (e.g., antibody or antibody binding protein) in the composition are high mannose glycoforms. In exemplary aspects, about 0.7% to about 3.0% of the recombinant glycosylated protein in the composition are high mannose glycoforms. In some aspects, the level of high mannose glycans in the antibody composition is about 0.7%, about 0.8%, about 0.9%, about 1.0%, about 1.1%, about 1.2%, about 1.3%, about 1.4%, about 1.5%, about 1.6%, about 1.7%, about 1.8%, about 1.9%, about 2.0%, about 2.1%, about 2.2%, about 2.3%, about 2.4%, about 2.5%, about 2.6%, about 2.7%, about 2.8%, about 2.9%, or about 3.0%.

In exemplary embodiments, the composition is combined with a pharmaceutically acceptable carrier, diluent or excipient. Accordingly, provided herein are pharmaceutical compositions comprising the recombinant glycosylated protein composition (e.g., the antibody composition or antibody binding protein composition) described herein and a pharmaceutically acceptable carrier, diluent or excipient. As used herein, the term “pharmaceutically acceptable carrier” includes any of the standard pharmaceutical carriers, such as a phosphate buffered saline solution, water, emulsions such as an oil/water or water/oil emulsion, and various types of wetting agents.

### Cell-Culture Medium

Provided herein is a cell culture medium comprising: (a) glycosylation-competent cells comprising an exogenous nucleotide sequence encoding an antibody; and (b) a culture medium comprising fucose at a concentration of about 0.1 g/L to about 1.0 g/L or about 0.17 g/L to about 1.0 g/L. The glycosylation-competent cells may be any cell described herein. In exemplary instances, the glycosylation-competent cells are not genetically modified to alter activity of an enzyme of the de novo pathway or the salvage pathway, optionally, wherein the glycosylation-competent cells are not genetically modified to knock-out a gene encoding GDP-keto-6-deoxymannonse-3,5-epimerase, 4-reductase. In exemplary embodiments, the culture medium further comprises glucose. In some aspects, the culture medium comprises glucose at a concentration less than about 10 g/L or less than about 9 g/L, e.g., about 6 g/L or less or about 0.5 g/L to about 4 g/L. In some aspects, the culture medium comprising the fucose. In exemplary aspects, the pH of the culture medium is about 6.85 to about 7.05, optionally, about 6.90 to about 7.00. In exemplary instances, the glycosylation-competent cells are not genetically modified to alter activity of an enzyme of the de novo pathway or the salvage pathway. For example, the glycosylation-competent cells are not genetically modified to knock-out a gene encoding GDP-keto-6-deoxymannonse-3,5-epimerase, 4-reductase. In exemplary aspects, the antibody is an IgG antibody, optionally, an IgG1 antibody. The IgG1 antibody in exemplary aspects is specific for a tumor associated antigen, e.g., CD20. In exemplary aspects, the cell culture medium does not comprise mannose.

### Modulation Methods

Methods of altering or modulating the levels of TAF glycans of a recombinant glycosylated protein (e.g., an antibody composition or antibody binding protein composition) produced by glycosylation-competent cells in a cell culture medium are further provided herein. In exemplary aspects, the method comprises (A) adding fucose to a cell culture medium comprising the glycosylation-competent cells to achieve a fucose concentration of about 0.1 g/L to about 1.0 g/L to decrease the level of TAF glycans; (B) adding glucose to a cell culture medium comprising the glycosylation-competent cells to achieve a glucose concentration less than about 10 g/L to increase the level of TAF glycans; or (C) both (A) and (B).

The present disclosure also provides methods of modulating the level of afucosylated glycans of a recombinant glycosylated protein (e.g., an antibody composition or antibody binding protein composition) produced by glycosylation-competent cells. In exemplary aspects, the methods comprise (A) adding fucose to a cell culture medium comprising the glycosylation-competent cells to achieve a fucose concentration of about 0.1 g/L to about 1.0 g/L to decrease the level of afucosylated glycans; (B) adding glucose to a cell culture medium comprising the glycosylation-competent cells to achieve a glucose concentration less than about 10 g/L to increase the level of afucosylated glycans; or (C) both (A) and (B).

Also provided are methods of modulating the level of high mannose glycans of a recombinant glycosylated protein (e.g., an antibody composition or antibody binding protein composition) produced by glycosylation-competent cells. In exemplary embodiments, the methods comprise adding glucose to a cell culture medium comprising the glycosylation-competent cells to achieve a glucose concentration less than about 10 g/L to increase the level of HM glycans.

Accordingly, in some exemplary embodiments, the methods of the disclosure relate to increasing the levels of TAF, HM, or afucosylated glycans of a protein, e.g., an antibody, produced by cells in a cell culture. In exemplary aspects, the levels of HM glycoforms of the recombinant glycosylated protein are increased, relative to the control cell culture. In exemplary aspects, the levels of one or more of Man5, Man6, Man7, Man8, and/or Man9 of the recombinant glycosylated protein are increased, relative to the control cell culture. In exemplary aspects, the levels of afucosylated glycoforms of the recombinant glycosylated protein are increased, relative to the control cell culture. In exemplary aspects, the levels of one or more of A1G0, A2G0, A2G1a, A2G1b, A2G2, and A1G1M5 of the recombinant glycosylated protein are increased, relative to the control cell culture. In exemplary aspects, the levels of one or more of A1G1a, G0[H3N4], G0[H4N4], G0[H5N4], and FO-N[H3N3] of the recombinant glycosylated protein are increased, relative to the control cell culture. In some aspects, the increase is an increase relative to the control cell culture, as determined by Hydrophilic Interaction Liquid Chromatography (HILIC). In some aspects, the increase is an increase relative to the control cell cultured as determined by methods known to one of skill in the art.

In some aspects, the methods of the disclosure increase the levels of TAF, HM, or afuco glycoform to any degree or level relative a control cell culture. For example, in some aspects, the increase provided by the methods of the disclosure is at least or about a 1% to about a 10% increase (e.g., at least or about a 1% increase, at least or about a 2% increase, at least or about a 3% increase, at least or about a 4% increase, at least or about a 5% increase, at least or about a 6% increase, at least or about a 7% increase, at least or about a 8% increase, at least or about a 9% increase, at least or about a 9.5% increase, at least or about a 9.8% increase, at least or about a 10% increase) relative a control cell culture. In exemplary embodiments, the increase provided by the methods of the disclosure is over 100%, e.g., 200%, 300%, 400%, 500%, 600%, 700%, 800%, 900% or even 1000% relative a control cell culture. In exemplary embodiments, the level of TAF, HM, or afuco glycoforms of the protein increases by at least about 1.5-fold, relative a control cell culture. In exemplary embodiments, the level of TAF, HM, or afuco glycoforms of the protein increases by at least about 2-fold, relative a control cell culture. In exemplary embodiments, the level of TAF, HM, or afuco glycoforms of the protein increases by at least about 3-fold, relative a control cell culture. In exemplary embodiments, the level of TAF, HM, or afuco glycoforms of the protein increases by at least about 4-fold or about 5-fold, relative to a control cell culture.

In exemplary aspects, the increased level of TAF glycoforms of the recombinant glycosylated protein is observed or observable or detected or detectable as early as the 1^{st} day after the fucose and/or glucose concentration is changed. In exemplary aspects, the increased level of TAF glycoforms of the recombinant glycosylated protein is observed or observable or detected or detectable as early as the 2^{nd} day post-change. In exemplary aspects, the increased level of TAF glycoforms of the recombinant glycosylated protein is observed or observable or detected or detectable as early as the 3^{rd} day post-change. In exemplary aspects, the increased level of TAF glycoforms of the recombinant glycosylated protein is observed or observable or detected or detectable as early as about the 4^{th} day post-change. In exemplary aspects, the increased level of TAF glycoforms of the recombinant glycosylated protein is observed or observable or detected or detectable after about the 5^{th} day post-change. In exemplary aspects, the increased level of TAF glycoforms of the protein is observed or observable or detected or detectable at the time the recombinant glycosylated protein is harvested from the cell culture.

In exemplary aspects, the increased level of TAF glycoforms of the recombinant glycosylated protein is observed for longer than about the 4^{th}, 5^{th}, or 6^{th} day of cell culture or beyond. In exemplary aspects, the increased level of TAF glycoforms of the recombinant glycosylated protein is observed for 7, 8, 9, 10, 11 or 12 days of cell culture (post-inoculation), or longer (e.g., about 2 weeks, about 3 weeks, about 4 weeks, about 1 month, about 2 months, about 3 months, about 6 months, or about 1 year). In exemplary aspects, the increased level of TAF glycoforms of the protein is observed at the time the protein is harvested from the cell culture.

In other aspects, the methods of the disclosure relate to decreasing the levels of TAF glycoforms of a protein produced by cells in a cell culture. In exemplary aspects, the levels of HM glycoforms of the recombinant glycosylated protein are decreased, relative to the control cell culture. In exemplary aspects, the levels of one or more of Man5, Man6, Man7, Man8, and/or Man9 of the recombinant glycosylated protein are decreased, relative to the control cell culture. In exemplary aspects, the levels of afucosylated glycoforms of the recombinant glycosylated protein are decreased, relative to the control cell culture. In exemplary aspects, the levels of one or more of A1G0, A2G0, A2G1a, A2G1b, A2G2, and A1G1M5 of the recombinant glycosylated protein are decreased, relative to the control cell culture. In exemplary aspects, the levels of one or more of A1G1a, G0[H3N4], G0[H4N4], G0[H5N4], and FO-N[H3N3] of the recombinant glycosylated protein are decreased, relative to the control cell culture. In exemplary aspects, the method is a method of decreasing the level of TAF glycoforms by about 1% to about 4% and the method comprises maintaining the glycosylation-competent cells in a first cell culture medium cell and increasing the fucose concentration to about 0.1 g/L to about 1.0 g/L. In some aspects, the decrease is a decrease relative to the control cell culture, as determined by HILIC. In some aspects, the decrease is a decrease relative to the control cell cultured as determined by methods known to one of skill in the art.

In some aspects, the methods of the disclosure decrease the level(s) of TAF, HM, or afuco glycoform to any degree or level relative a control cell culture. For example, the decrease provided by the methods of the disclosure is at least or about a 0.1% to about a 1% decrease (e.g., at least or about a 0.1% decrease, at least or about a 0.2% decrease, at least or about a 0.3% decrease, at least or about a 0.4% decrease, at least or about a 0.5% decrease, at least or about a 0.6% decrease, at least or about a 0.7% decrease, at least or about a 0.8% decrease, at least or about a 0.9% decrease, at least or about a 0.95% decrease, at least or about a 0.98% decrease, at least or about a 1.0% decrease) relative to the level of a control cell culture. In exemplary embodiments, the decrease provided by the methods of the disclosure is over about 100%, e.g., about 200%, about 300%, about 400%, about 500%, about 600%, about 700%, about 800%, about 900% or even about 1000% relative to the level of a control cell culture. In exemplary embodiments, the level of TAF, HM, or afuco glycoforms of the protein decreases by at least or about 1.5-fold, relative to a control cell culture. In exemplary embodiments, the level of TAF, HM, or afuco glycoforms of the protein decreases by at least about 2-fold, relative to a control cell culture. In exemplary embodiments, the level of TAF, HM, or afuco glycoforms of the protein decreases by at least about 3-fold, relative to a control cell culture. In exemplary embodiments, the level of TAF, HM, or afuco glycoforms of the protein decreases by at least about 4-fold or by at least about 5-fold, relative to a control cell culture.

In exemplary aspects, the decreased level of TAF glycoforms of the recombinant glycosylated protein is observed or observable or detected or detectable as early as about the 1^{st} day post-inoculation. In exemplary aspects, the decreased level of TAF glycoforms of the recombinant glycosylated protein is observed or observable or detected or detectable as early as about the 2^{nd} day post-inoculation. In exemplary aspects, the decreased level of TAF glycoforms of the recombinant glycosylated protein is observed or observable or detected or detectable as early as about the 3^{rd} day post-inoculation. In exemplary aspects, the decreased level of TAF glycoforms of the recombinant glycosylated protein is observed or observable or detected or detectable as early as about the 4^{th} day post-inoculation. In exemplary aspects, the decreased level of TAF glycoforms of the recombinant glycosylated protein is observed or observable or detected or detectable after about the 5^{th} day post-inoculation. In exemplary aspects, the decreased level of TAF glycoforms of the recombinant glycosylated protein is observed or observable or detected or detectable at about the time the protein is harvested from the cell culture.

In exemplary aspects, the decreased level of TAF glycoforms of the protein is observed for longer than about the 4^{th}, about the 5^{th}, or about the 6^{th} day of cell culture or beyond the initial cell culture period. In exemplary aspects, the decreased level of TAF glycoforms of the protein is observed for about 7, about 8, about 9, about 10, about 11 or about 12 days of cell culture (post-inoculation), or longer (e.g., about 2 weeks, about 3 weeks, about 4 weeks, about 1 month, about 2 months, about 3 months, about 6 months, or about 1 year). In exemplary aspects, the decreased level of TAF glycoforms of the protein is observed at about the time the protein is harvested from the cell culture.

With regard to the methods of the disclosure, the modulation, increase or decrease affected by such methods are relative to a “control” or a “control cell culture.” In exemplary aspects, the control is the level of TAF glycoforms of the protein when the steps of the inventive method are not carried out. In exemplary aspects, the control is the level of TAF glycoforms of the protein when a known method of recombinant production is carried out. In exemplary aspects, the control is the level of TAF glycoforms when a known glucose or fucose concentration is maintained during recombinant production. As used herein, the term “control cell culture” means a cell culture maintained in the same manner as the cell culture on which the steps of the inventive method are carried out (e.g., cell culture of the disclosed methods) except for the fucose/glucose concentration. In exemplary aspects, the control cell culture is a cell culture maintained at known operational or standard parameters, including a control fucose/glucose concentration. As used herein, the term “control fucose concentration” or “control glucose concentration” may refer to a known operational fucose/glucose concentration, e.g., a fucose/glucose concentration of a cell culture maintained at a first time point or at a time point before carrying out the methods of the disclosure. In exemplary aspects, a control fucose/glucose concentration is a fucose/glucose concentration of a cell culture for which the TAF levels are known or determined.

In exemplary aspects of the modulating methods of the disclosure, the glycosylation-competent cells are not genetically modified to alter activity of an enzyme of the de novo pathway or the salvage pathway. Optionally, the glycosylation-competent cells are not genetically modified to knock-out a gene encoding GDP-keto-6-deoxymannonse-3,5-epimerase, 4-reductase.

In exemplary aspects, after the methods of the disclosure are carried out, the level of TAF glycans in the antibody composition is less than about 10%, e.g., about 2% to about 6%, about 2% to about 5%, or about 2% to about 4%.

In exemplary aspects, after the methods of the disclosure are carried out, the level of high mannose glycans in the antibody composition is less than about 3.5%, optionally, about 0.7% to about 3.0%.

In exemplary aspects, after the methods of the disclosure are carried out, the level of afucosylated glycans in the antibody composition is less than about 3.5%, optionally, about 0.8% to about 2.8%.

With regard to the modulating methods described herein comprising adding fucose, the final fucose concentration of the cell culture medium, in various aspects, is about 0.17 g/L to about 1.0 g/L, or about 0.2 g/L to about 0.5 g/L. However, any of the fucose concentrations described herein are contemplated.

With regard to the modulating methods described herein comprising adding glucose to the cell culture medium, in some aspects, the glucose is added according to a glucose feeding schedule that achieves an average glucose concentration of about 10 g/L or less or about 9 g/L or less. In some aspects, the average glucose concentration is less than about 6.0 g/L, optionally, less than about 4.0 g/L. In some instances, the average glucose concentration is based on the fucose concentration of the cell culture medium. For example, the average glucose concentration may be calculated based on the Formula I:

$$T = 3.354 - 1.388 F + 0.111 G + [ F - 0.43 \; 75 ] \times [ 1.9527 ( F - 0.4375 ) ] \qquad Formula 1$$

wherein T is the targeted % TAF glycans and is 2.5% to about 6%, about 2.75% to about 5.5%, or about 3% to about 5%, F is the concentration (g/L) of fucose in the medium, and G is the average glucose concentration (g/L).

The present disclosure additionally provides methods of modulating (reducing or increasing) the level of afucosylated glycans of a recombinant glycosylated protein composition (e.g., an antibody composition or antibody binding protein composition) produced by glycosylation-competent cells. In exemplary embodiments, the method comprises reducing the pH of the cell culture medium by about 0.03 to about 1.2 (e.g., 0.05 to about 1.0) to reduce the level of afucosylated glycans of a recombinant glycosylated protein composition (e.g., an antibody composition or antibody binding protein composition) by about 0.5% to about 2% (e.g., 0.5% to about 1.5%, 0.5% to about 1.0%, 1.0% to about 2%, 1.5% to about 2.0%) or increasing the pH of the cell culture medium by about 0.03 to about 1.2 (e.g., 0.05 to about 1.0) to increase the level of afucosylated glycans of a recombinant glycosylated protein composition (e.g., an antibody composition or antibody binding protein composition) by about 0.5% to about 2% (e.g., 0.5% to about 1.5%, 0.5% to about 1.0%, 1.0% to about 2%, 1.5% to about 2.0%). In exemplary aspects, the method comprises reducing the pH of the cell culture medium by about 0.05 to about 1.2 (e.g., 0.06, 0.07, 0.08, 0.09, 1.1, 1.11, 1.12, 1.13, 1.14, 1.15, 1.16, 1.17, 1.18, 1.19, 1.20), to reduce the level of afucosylated glycans of the recombinant glycosylated protein composition (e.g., an antibody composition or antibody binding protein composition) by about 1% to about 2% (e.g., 1.0% to 1.5%, 1.5% to 2.0%) or increasing the pH of the cell culture medium by about 0.05 to about 1.2 (e.g., 0.06, 0.07, 0.08, 0.09, 1.1, 1.11, 1.12, 1.13, 1.14, 1.15, 1.16, 1.17, 1.18, 1.19, 1.20), to increase the level of afucosylated glycans of the recombinant glycosylated protein composition (e.g., an antibody composition or antibody binding protein composition) by about 1% to about 2% (e.g., 1.0% to 1.5%, 1.5% to 2.0%). In various instances, the method comprises reducing the pH of the cell culture medium by about 0.03 to about 0.07 (e.g., 0.03, 0.04, 0.05, 0.06, 0.07) to reduce the level of afucosylated glycans of the recombinant glycosylated protein composition (e.g., an antibody composition or antibody binding protein composition) by about 0.5% to about 1.1% (e.g., 0.05%, 0.06%, 0.07%, 0.08%, 0.09%. 1.0%, 1.01%, 1.02%, 1.03%, 1.04%, 1.05%, 1.06%, 1.07%, 1.08%, 1.09%, 1.10%) or increasing the pH of the cell culture medium by about 0.03 to about 0.07 (e.g., 0.03, 0.04, 0.05, 0.06, 0.07) to increase the level of afucosylated glycans of the recombinant glycosylated protein composition (e.g., an antibody composition or antibody binding protein composition) by about 0.5% to about 1.1% (e.g., 0.05%, 0.06%, 0.07%, 0.08%, 0.09%. 1.0%, 1.01%, 1.02%, 1.03%, 1.04%, 1.05%, 1.06%, 1.07%, 1.08%, 1.09%, 1.10%).

Methods of reducing the level of afucosylated glycans of a recombinant glycosylated protein composition (e.g., an antibody composition or antibody binding protein composition) produced by glycosylation-competent cells by about 1% to about 2% are also provided herein. In exemplary embodiments, the method comprises reducing the pH of the cell culture medium by about 0.05 to about 1.2. Optionally, the method comprises reducing the pH by about 0.05 to about 0.07 for a reduction in afucosylated glycans of about 1% or reducing the pH by about 0.09 to about 1.2 for a reduction in afucosylated glycans of more than about 1.5%. In various aspects, the method comprises culturing the cells at a pH between about 7.10 to about 7.20, optionally about 7.12 to about 7.19.

Methods of reducing the level of afucosylated glycans of a recombinant glycosylated protein composition (e.g., an antibody composition or antibody binding protein composition) produced by glycosylation-competent cells by about 0.5% to about 1.1% are furthermore provided. In exemplary embodiments, the method comprises reducing the pH of the cell culture medium by about 0.03-0.07. In various aspects, the method comprises reducing the pH by about 0.03 to about 0.06 for a reduction in afucosylated glycans of about 0.8%. In some aspects, the method comprises reducing the pH by about 0.05 to about 0.07 for a reduction in afucosylated glycans of about 1%. In various instances, the method comprises culturing the cells at a pH between about 7.05 to about 7.15, optionally about 7.07 to about 7.13.

Methods of increasing the level of afucosylated glycans of a recombinant glycosylated protein composition (e.g., an antibody composition or antibody binding protein composition) produced by glycosylation-competent cells by about 1% to about 2% are provided by the present disclosure. In exemplary embodiments, the method comprises increasing the pH of the cell culture medium by about 0.05 to about 1.2. Optionally, the method comprises increasing the pH by about 0.05 to about 0.07 for a reduction in afucosylated glycans of about 1% or increasing the pH by about 0.09 to about 1.2 for a reduction in afucosylated glycans of more than about 1.5%. In some aspects, the method comprises culturing the cells at a pH between about 7.10 to about 7.20, optionally about 7.12 to about 7.19.

The present disclosure also provides methods of increasing the level of afucosylated glycans of a recombinant glycosylated protein composition (e.g., an antibody composition or antibody binding protein composition) produced by glycosylation-competent cells by about 0.5% to about 1.1%. In exemplary embodiments, the method comprises increasing the pH of the cell culture medium by about 0.03-0.07 or increasing the pH by about 0.03 to about 0.06 for a reduction in afucosylated glycans of about 0.8% or increasing the pH by about 0.05 to about 0.07 for a reduction in afucosylated glycans of about 1%. In various instances, the method comprises culturing the cells at a pH between about 7.05 to about 7.15, optionally about 7.07 to about 7.13.

In any of the foregoing methods, the pH of the cell culture medium throughout the culture is greater than 7.0, optionally, higher than 7.05 and less than 7.2. In some aspects, the level of afucosylated glycans in the recombinant glycosylated protein composition (e.g., an antibody composition or antibody binding protein composition) is less than about 10%, e.g., about 6.2% to about 8.4%. In any of the foregoing methods, the temperature changes by less than 2 degrees C. during the culture period. For example, in some aspects, the temperature of the culture changes by not more than 1.5 or 1.0 degrees C. In any of the foregoing methods, the cell culture medium does not comprise any detectable amounts of manganese or betaine. The cell culture medium in some aspects comprises about 0.10 g/L to about 1.0 g/L fucose, optionally, about 0.17 to about 1.0 g/L fucose. Optionally, fucose is present in the culture medium at a concentration less than about 0.75 g/L, less than about 0.6 g/L, or about 0.2 g/L to about 0.5 g/L. The addition or presence of fucose in the culture medium may be in accordance with any of the teachings provided herein. In exemplary aspects, the glycosylation-competent cells are not genetically modified to alter activity of an enzyme of the de novo pathway or the salvage pathway. In any of the foregoing methods, glucose is added to the cell culture medium according to a glucose feeding schedule that achieves an average glucose concentration of about 10 g/L or less. The glucose may be added in accordance with any of the teachings provided herein.

As discussed above, TAF glycans is the sum of HM glycans and afucosylated glycans. Accordingly, the presently disclosed methods of modulating afucosylated glycans will in various instances modulate the level of TAF glycans of a recombinant glycosylated protein composition (e.g., an antibody composition or antibody binding protein composition). Accordingly, methods of modulating the level of TAF glycans of a recombinant glycosylated protein composition (e.g., an antibody composition or antibody binding protein composition) produced by glycosylation-competent cells are provided herein. The method in exemplary aspects comprises modulating the level of afucosylated glycans of the recombinant glycosylated protein composition (e.g., an antibody composition or antibody binding protein composition) in accordance with a presently disclosed method of modulating the level of afucosylated glycans. In exemplary embodiments, the method of modulating TAF glycans comprises reducing the level of afucosylated glycans of the recombinant glycosylated protein composition (e.g., an antibody composition or antibody binding protein composition) in accordance with a presently disclosed method of reducing the level of afucosylated glycans or increasing the level of afucosylated glycans of the recombinant glycosylated protein composition (e.g., an antibody composition or antibody binding protein composition) in accordance with a presently disclosed method of increasing afucosylated glycans.

A method of producing an antibody composition, wherein the level of afucosylated glycans in the antibody composition is about 6.2% to about 8.4%, is furthermore provided by the present disclosure. In exemplary embodiments, the method comprises maintaining glycosylation-competent cells in a cell culture medium at a pH higher than 7.05 and lower than 7.2,
wherein, optionally:
   (A) the pH of the cell culture medium changes by less than 0.15 (optionally by less than 0.10) during the culture period or
   (B) the temperature of the cell culture medium changes by not more than 2 degrees C. or
   (C) the method does not comprise culturing the cells in a cell culture medium comprising manganese or betaine or
   (D) a combination of two or three of (A), (B), and (C).

In various aspects, the pH is maintained at a pH of about 7.07 to about 7.19 (e.g. 7.08, 7.09, 7.10, 7.11, 7.12, 7.13, 7.14, 7.15, 7.16, 7.17, 7.18, or 7.19) during the culture period, optionally, wherein the pH is maintained at about 7.07 or higher and below 7.10, or about 7.10 or higher and below 7.15, or about 7.15 or higher up to about 7.19. In various aspects, the level of afucosylated glycans in the antibody composition is less than about 10%, optionally, about 6.2% to about 8.4%. In various instances, the temperature changes by less than 2 degrees C. during the culture period, optionally, the temperature of the culture changes by not more than 1.5 or 1.0 degrees C. In various aspects, the cell culture medium does not comprise any detectable amounts of manganese or betaine. In exemplary aspects, the cell culture medium comprises about 0.10 g/L to about 1.0 g/L fucose, optionally, about 0.17 to about 1.0 g/L fucose, optionally, fucose is present in the culture medium at a concentration less than about 0.75 g/L, less than about 0.6 g/L, or about 0.2 g/L to about 0.5 g/L. In some aspects, the glycosylation-competent cells are not genetically modified to alter activity of an enzyme of the de novo pathway or the salvage pathway, and in some aspects, glucose is added to the cell culture medium according to a glucose feeding schedule that achieves an average glucose concentration of about 10 g/L or less.

### Exemplary Embodiments

The disclosure provides methods of producing a recombinant glycosylated protein composition (e.g., an antibody composition or antibody binding protein composition). In exemplary embodiments, the method comprises maintaining glycosylation-competent cells in a cell culture medium comprising fucose and/or glucose at a specific concentration as described herein, depending on the level of TAF glycoforms desired. In exemplary embodiments, the level of TAF glycoforms in the recombinant glycosylated protein composition (e.g., antibody composition or antibody binding protein composition) is less than or about 10% and, in exemplary aspects, the method comprises maintaining glycosylation-competent cells in a cell culture medium comprising fucose, wherein fucose is present in the culture medium at a concentration between about 0.17 g/L and about 1.0 g/L. In exemplary embodiments, the level of TAF glycoforms in the recombinant glycosylated protein composition (e.g., antibody composition or antibody binding protein composition) is less than or about 10% and, in exemplary aspects, the method comprises maintaining glycosylation-competent cells in a cell culture medium comprising fucose, wherein fucose is present in the culture medium at a concentration between about 0.1 g/L and about 1.0 g/L, and wherein the glycosylation-competent cells are not genetically modified to alter activity of an enzyme of the de novo pathway or the salvage pathway. The disclosure also provides methods of producing a recombinant glycosylated protein composition (e.g., an antibody composition or antibody binding protein composition) comprising maintaining glycosylation-competent cells in a cell culture medium comprising fucose and glucose, wherein fucose is present in the culture medium at a concentration of about 0.1 g/L to about 1.0 g/L and adding glucose to the cell culture medium according to a glucose feeding schedule that achieves an average glucose concentration of about 10 g/L or less. In exemplary aspects, fucose is present in the culture medium at a concentration less than about 0.75 g/L, optionally less than about 0.6 g/L. In exemplary instances, fucose is present in the culture medium at a concentration of about 0.2 g/L to about 0.5 g/L. In some aspects, fucose is present in the culture medium the entire duration the glycosylation-competent cells are maintained in cell culture. In exemplary instances, the method of producing a recombinant glycosylated protein composition (e.g., an antibody composition or antibody binding protein composition) comprises maintaining the glycosylation-competent cells in a first cell culture medium for an initial time period and subsequently maintaining the glycosylation-competent cells in a second cell culture medium, wherein the first cell culture medium does not comprise fucose at a concentration of about 0.1 g/L to about 1.0 g/L and the second cell culture medium comprises fucose at a concentration of about 0.1 g/L to about 1.0 g/L. In some instances, the initial time period is about 24 to about 72 hours. In alternative aspects, the initial time period is about or greater than about 72 hours but less than or about 156 hours. In some aspects, fucose is added to the first culture medium on the 6th day post-cell culture inoculation to obtain the second cell culture medium. the concentration of fucose fluctuates by about 0.2 g/L or less (e.g., 0.1 g/L or less) during the time the glycosylation-competent cells are maintained in the cell culture medium comprising fucose. In some aspects, the cell culture medium comprises an initial glucose concentration for an initial time period. Optionally, the initial glucose concentration is about 1 g/L to about 15 g/L, e.g., about 12 g/L±1 g/L. In exemplary aspects, the method further comprises adding glucose to the cell culture medium according to a glucose feeding schedule. In exemplary aspects, the glucose feeding schedule is initiated at about 4 to about 6 days post-cell culture inoculation. For example, the glucose feeding schedule may be initiated at about 6 days post-cell culture inoculation. In exemplary instances, the glucose feeding schedule achieves an average glucose concentration of about 10 g/L or less (e.g., about 9 g/L or less, about 6 g/L or less) in the cell culture medium, about 0.5 g/L to about 4 g/L). In exemplary instances, the glucose feeding schedule achieves an average glucose concentration based on the concentration of fucose in the cell culture medium. In exemplary aspects, the average glucose concentration is calculated based on Formula I:

$$T = 3.354 - 1.388 F + 0.111 G + [ F - 0.43 \; 75 ] \times [ 1.9527 ( F - 0.4375 ) ] \qquad ( Formula 1 )$$

wherein T is the targeted % total afucosylated (TAF) glycans in the antibody composition and is about 2.5% to about 6%, about 2.75% to about 5.5%, or about 3% to about 5%, F is the concentration (g/L) of fucose in the medium, and G is the average glucose concentration (g/L) in the medium. In exemplary aspects, (i) the concentration of fucose is about 0.2±0.1 g/L and the average glucose concentration is about 2 to about 4 g/L; (ii) the concentration of fucose is about 0.5±0.1 g/L and the average glucose concentration is about 3 to about 6 g/L; or (iii) the concentration of fucose is about 0.75±0.1 g/L and the average glucose concentration is about 4.5 to about 9 g/L. In exemplary aspects, the pH of the cell culture medium is about 6.85 to about 7.05 (e.g., about 6.90 to about 7.00). In some aspects, the glycosylation-competent cells are not genetically modified to alter activity of an enzyme of the de novo pathway or the salvage pathway. For example, the glycosylation-competent cells are not genetically modified to knock-out a gene encoding GDP-keto-6-deoxymannonse-3,5-epimerase, 4-reductase. In exemplary instances, the level of total afucosylated (TAF) glycans in the antibody composition is less than about 10% (e.g., about 2% to about 6%, about 2% to about 5%, about 2% to about 4%). In exemplary instances, the level of high mannose glycans in the antibody composition is less than about 3.5% (e.g., about 0.7% to about 3.0%). In exemplary instances, the level of afucosylated glycans in the antibody composition is less than about 3.5% (e.g., about 0.8% to about 2.8%). In some aspects, the glycosylation-competent cells produce IgG antibodies, optionally, IgG1 antibodies. In some aspects, the IgG1 antibodies are specific for a tumor-associated antigen (e.g., CD20). In exemplary aspects, the culture medium does not comprise mannose.

A cell culture medium comprising: (a) glycosylation-competent cells comprising an exogenous nucleic acid encoding an antibody; and (b) a culture medium comprising fucose at a concentration of about 0.1 g/L to about 1.0 g/L or about 0.17 g/L to about 1.0 g/L are provided. In exemplary aspects, the glycosylation-competent cells are not genetically modified to alter activity of an enzyme of the de novo pathway or the salvage pathway, optionally, wherein the glycosylation-competent cells are not genetically modified to knock-out a gene encoding GDP-keto-6-deoxymannonse-3,5-epimerase, 4-reductase. In exemplary aspects, the culture medium further comprises glucose at a concentration less than about 10 g/L, e.g., less than about 9 g/L, less than about 6 g/L, or about 0.5 g/L to about 4 g/L. In exemplary aspects, the pH of the culture medium is about 6.85 to about 7.05, e.g., about 6.9 to about 7.0. In some aspects, the cell culture medium does not comprise mannose. In exemplary aspects, the antibody is an IgG antibody, e.g., an IgG1 antibody. In exemplary instances, the IgG1 antibody is specific for a tumor-associated antigen, e.g., CD20.

Methods of altering or modulating the level of TAF glycans of a recombinant glycosylated protein composition (e.g., an antibody composition or antibody binding protein composition) produced by glycosylation-competent cells in a cell culture medium are further provided herein. In exemplary aspects, the method comprises (A) adding fucose to a cell culture medium comprising the glycosylation-competent cells to achieve a fucose concentration of about 0.1 g/L to about 1.0 g/L to decrease the level of TAF glycans; (B) adding glucose to a cell culture medium comprising the glycosylation-competent cells to achieve a glucose concentration less than about 10 g/L to increase the level of TAF glycans; or (C) both (A) and (B). Also provided are methods of modulating the level of afucosylated glycans of a recombinant glycosylated protein composition (e.g., an antibody composition or antibody binding protein composition) produced by glycosylation-competent cells. In exemplary embodiments, the method comprises (A) adding fucose to a cell culture medium comprising the glycosylation-competent cells to achieve a fucose concentration of about 0.1 g/L to about 1.0 g/L to decrease the level of afucosylated glycans; (B) adding glucose to a cell culture medium comprising the glycosylation-competent cells to achieve a glucose concentration less than or about 10 g/L to increase the level of afucosylated glycans; or (C) both (A) and (B). The present disclosure further provides a method of modulating the level of high mannose glycans of a recombinant glycosylated protein composition (e.g., an antibody composition or antibody binding protein composition) produced by glycosylation-competent cells. In exemplary embodiments, the method comprises adding glucose to a cell culture medium comprising the glycosylation-competent cells to achieve a glucose concentration less than about 10 g/L to increase the level of HM glycans. In exemplary aspects of the methods of modulating, the glycosylation-competent cells are not genetically modified to alter activity of an enzyme of the de novo pathway or the salvage pathway. For example, the glycosylation-competent cells are not in some aspects, genetically modified to knock-out a gene encoding GDP-keto-6-deoxymannonse-3,5-epimerase, 4-reductase. In some aspects, the level of TAF glycans in the antibody composition is less than or about 10% (e.g., about 2% to about 6%, about 2% to about 5%, about 2% to about 4%). In some aspects, the level of high mannose glycans in the antibody composition is less than or about 3.5% (e.g., about 0.7% to about 3.0%). In some aspects, the level of afucosylated glycans in the antibody composition is less than or about 3.5% (e.g., about 0.8% to about 2.8%). In exemplary aspects, the fucose concentration is about 0.17 g/L to about 1.0 g/L, optionally, about 0.2 g/L to about 0.5 g/L. In some aspects, the method further comprises adding glucose to the cell culture medium according to a glucose feeding schedule that achieves an average glucose concentration of about 10 g/L (e.g., less than about 9.0 g/L, less than about 6.0 g/L, less than about 4.0 g/L). In some instances, the average glucose concentration is based on the fucose concentration of the cell culture medium. For example, in some aspects, the average glucose concentration is calculated based on Formula I:

$$T = 3.354 - 1.388 F + 0.111 G + [ F - 0.43 \; 75 ] \times [ 1.9527 ( F - 0.4375 ) ] \qquad Formula 1$$

wherein T is the targeted % total afucosylated (TAF) glycans in an antibody composition and is about 2.5% to about 6%, about 2.75% to about 5.5%, or about 3% to about 5%, F is the concentration (g/L) of fucose in the medium, and G is the average glucose concentration (g/L).

The following examples are given merely to illustrate the present disclosure and not in any way to limit its scope.

### EXAMPLES

### Example 1

This example describes the methods carried out and materials used in the experiments of Example 2.

### Cell Lines, Cell Culture and Media

All experiments were performed using a clone expressing an antibody comprising a light chain comprising SEQ ID NO. 1 and a heavy chain comprising SEQ ID NO: 2. All experiments were performed using a separate vial of cells cultured for 25 days. The following parameters were held constant: duration (12 days), dissolved oxygen (48 mm Hg to 74 mm Hg), pH (6.85 to 7.05), agitation (350 RPM, 20 W/m³), temperature (36.0° C.).

### Hydrophilic Interaction Liquid Chromatography (HILIC) Glycan Map

The glycan map of enzymatically released N-linked glycans was determined using HILIC. Briefly, glycans were incubated with a solution comprising PNGase F and a sodium phosphate buffer (pH 7.5) for ˜2 hours at ˜37° C. A labeling solution comprising 2-aminobenzoic acid (2-AA) and sodium cyanoborohydride was then added to the PNGase F-treated glycans and the mixture was incubated for ˜80° C. for about 75 minutes. After incubation, the mixtures were centrifuged to obtain a pellet of precipitated protein. Supernatants were collected and placed in vials.

The glycans were separated by HILIC, in line with a fluorescence detector: Glycans were injected and bound to the column in high organic conditions (Mobile Phase A and Mobile Phase B were ammonium formate and acetonitrile, respectively) and then eluted with an increasing gradient of an aqueous ammonium formate buffer. High resolution was achieved using a 1.7 μm small particle column format and 150 mm column length. The total run time, including column re-equilibration was 155 minutes.

### Example 2

This example demonstrates the effects of increasing glucose and fucose levels in the culture medium on TAF %.

Cells expressing an antibody comprising a light chain of SEQ ID NO: 1 and a heavy chain of SEQ ID NO: 2 were added to a bioreactor containing one of three culture media: a control culture medium, a first test culture medium, and a second test culture medium. The first test culture medium was identical to the control culture medium, except that it contained twice the amount of glucose, and the second test culture medium was the control culture medium with 0.5 g/L fucose. Each of the culture media lacked mannose. The cell culture was maintained for 12 days at a pH between 6.85 and 7.05.

Media samples were periodically taken from the bioreactors for measurement of glucose concentration, TAF levels and ADCC levels. TAF and/or afucosylated (Afuc) glycan levels were assayed via an HILIC N-glycan mapping procedure, and the ability to stimulate ADCC was tested using an in vitro assay. The results are shown in TABLE 1 below.

TABLE 1

| Culture Medium | TAF level (%) | ADCC (%) |
|---|---|---|
| Targeted Range | 2.0-4.2% | 69-97% |
| Control | 4.00 ± 0.23% | ~100% |
| First Test | 5.87 ± 0.23% | 152 ± 15% |
| Second Test | ~3.4% | ~85% |

ADCC levels are expressed as % ages relative to the ADCC level achieved with the control, which is a commercially-available antibody having the same amino acid sequence.

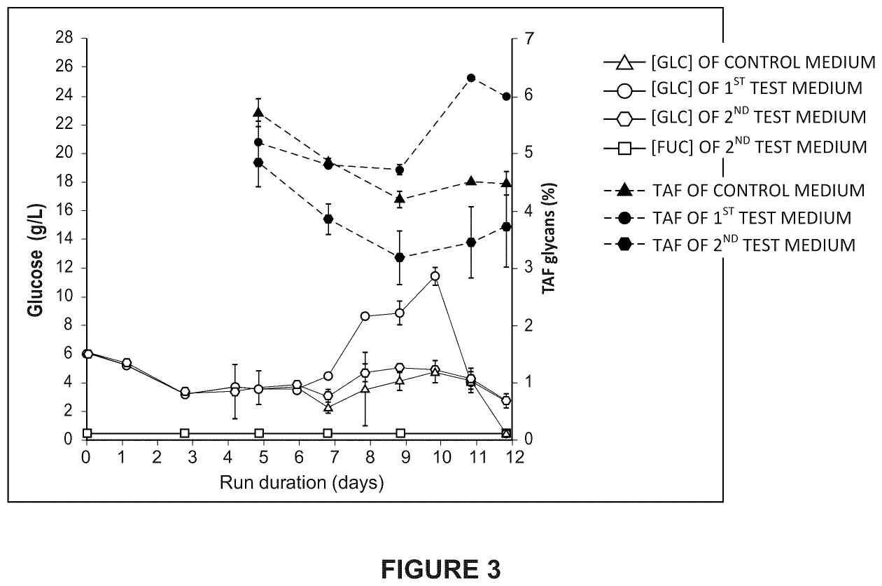

Glucose concentrations were determined throughout the culture run and these measurements were plotted as a function of time and in relation to TAF levels of the antibodies produced in each cell culture medium type. The results are shown in FIG. 3. As shown in this figure, the antibodies produced by cells cultured in the first test culture demonstrated an increase in TAF, which increase corresponded with the increase in glucose levels in the cell culture. These results (and the results of TABLE 1) suggest that glucose can affect TAF levels and ADCC.

The antibodies produced by cells cultured in the second test culture medium comprising 0.5 g/L fucose exhibited ˜1% decrease in TAF (FIG. 3). As shown in FIG. 3, the fucose concentration did not change much over the 12-day run, likely because fucose uptake by cells was negligible.

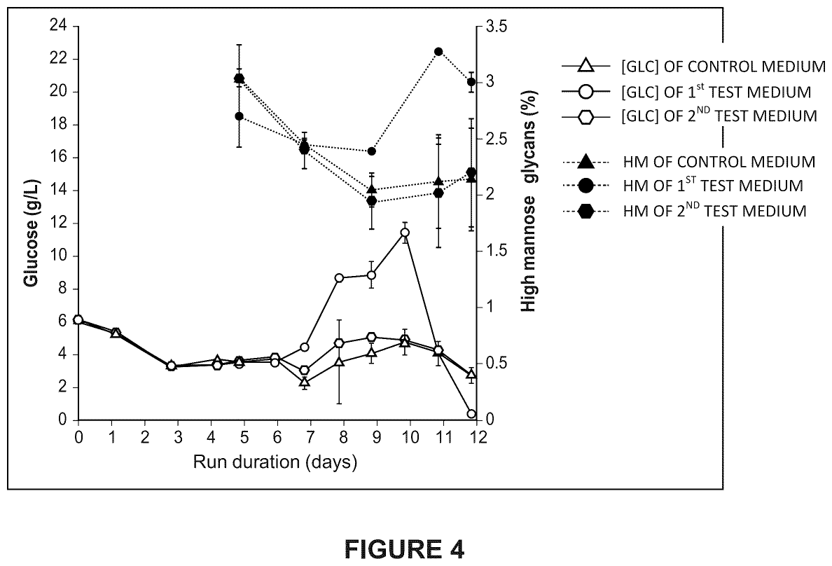

Further analysis of the separate glycan species of the antibodies produced in each of the different culture media was carried out. Interestingly, as shown in FIG. 4, the % high mannose (HM) glycans increased when the cells were cultured in the first test medium which contained 2-times the amount of glucose compared to control medium. This increase in % HM glycans was not achieved by cells cultured in the second test medium containing fucose. As shown in FIG. 4, the % HM glycans of antibodies produced by cells cultured in the second test medium containing fucose was about the same as the % HM glycans of the antibodies produced by cells cultured in the control medium.

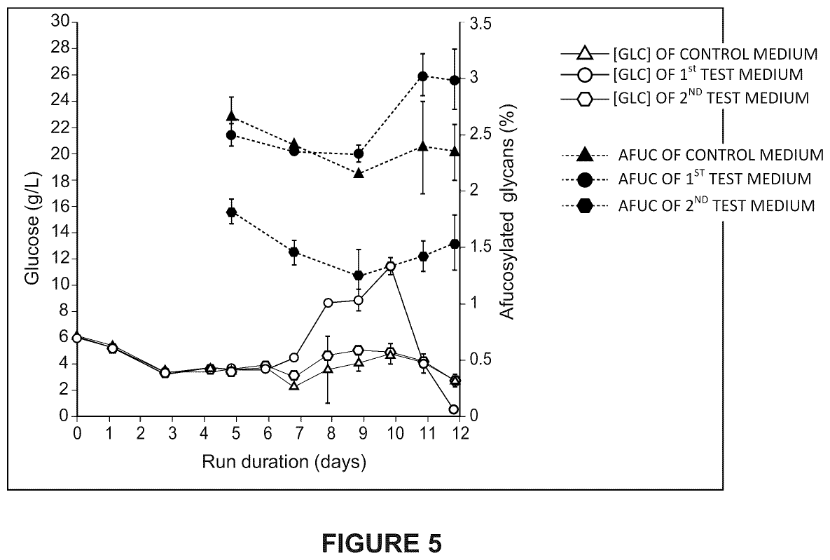

The effects of culturing in medium containing twice the amount of glucose (first test medium) or containing fucose (second test medium) on the % afucosylated glycans was similar to those effects on the % TAF glycans. As shown in FIG. 5, cells cultured in the first test medium comprising the higher glucose concentration demonstrated an increase in afucosylated glycans, whereas cells cultured in the second test medium comprising fucose produced antibodies with decreased afucosylated glycans.

This example demonstrated that glucose and fucose are levers that can be used to modulate high mannose and afucosylated glycan levels as well as impact ADCC.

### Example 3

This example demonstrates additional studies demonstrating glucose and fucose as levers for the modulation of TAF levels and ADCC.

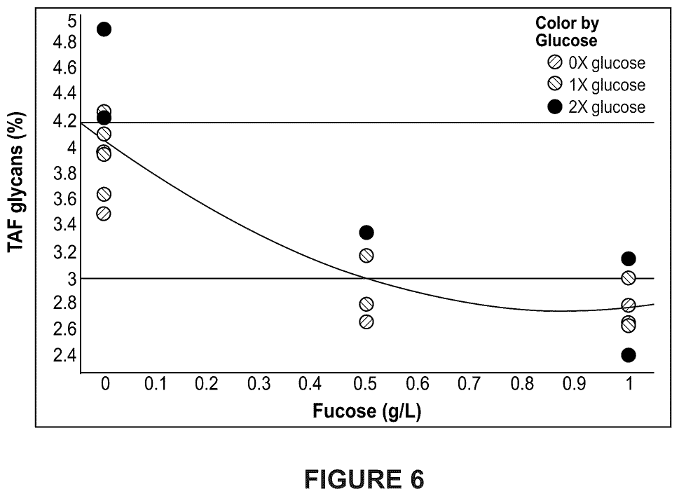

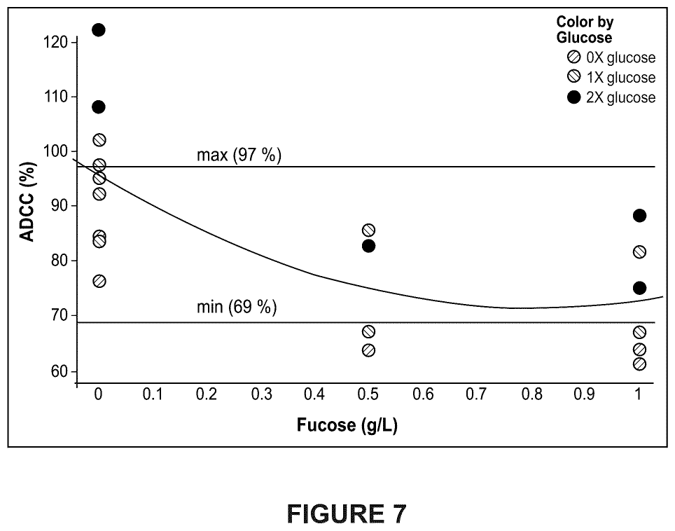

A follow-up multivariate full factorial experiment was designed to (1) elucidate the main, interaction and quadratic effects of fucose and glucose variables and (2) find amounts for these variables that would lead to modified TAF glycan and ADCC levels. Fucose was evaluated in culture media at concentrations of 0 g/L, 0.5 g/L and 1 g/L. Glucose was fed at 0×, 1× (control) and 2× rates. 0× meant that there was no glucose stock solution added to the cultures and this translated to a residual glucose concentration of ˜1g/L after Day 6 of cell culture. Glucose was supplied only through the media, which contained 12 g/L of the sugar. At 1× feeding, glucose was maintained at an average concentration of 3±1 g/L after glucose feed initiation. In 2× feeding, the average glucose levels were maintained at 6±1 g/L. The results indicate that fucose concentrations can be changed to impact the TAF levels (FIG. 6) and ADCC levels (FIG. 7).

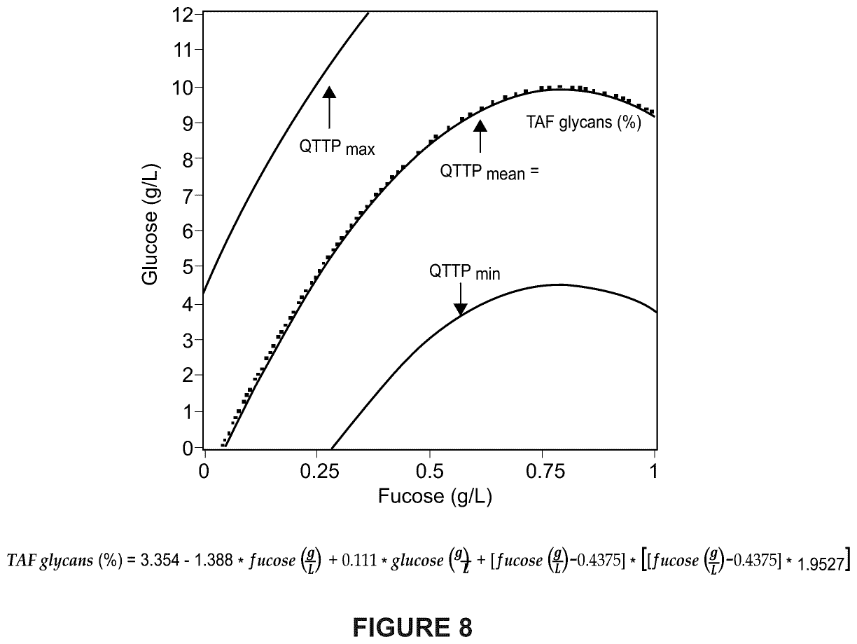

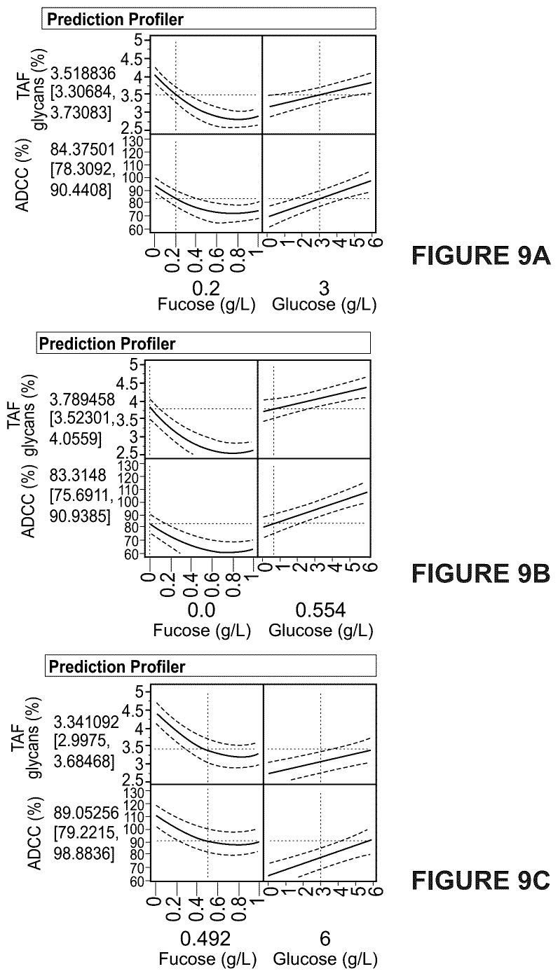

In these experiments, glucose concentration was controlled at 3±1 g/L post feed initiation (i.e., after Day 6). If glucose concentrations exceeded 4 g/L, no additional glucose was added and the cells were to rely on residual glucose in the bioreactor and glucose coming through the perfusion media until the glucose level fell within the control range. Based on these experiments, TAF values were predicted for the different fucose and glucose concentrations, according to a model shown in FIG. 8. The model demonstrates that the Quality Target Product Profile (QTTP) may be achieved upon culturing the cells in a culture medium comprising about 0.1 g/L to about 1.0 g/L fucose and/or about 0.5 g/L to about 4.0 g/L glucose. TAF values were predicted for the following different fucose and glucose concentrations utilizing this model: 0.2 g/L fucose and 3 g/L glucose, 0 g/L fucose and 0.554 g/L glucose; and 0.492 g/L fucose and 6 g/L glucose. FIGS. 9A-9C. These results suggested that are several ways to arrive at the desired TAF levels and ADCC levels.

To confirm the predictions of FIGS. 9A-9C, additional experiments were carried out. A summary of the experiments is provided in TABLE 2.

TABLE 2

| | Fucose | Glucose | TAF (95% | TAF | ADCC | ADCC |
|---|---|---|---|---|---|---|
| Condition | (g/L) | (g/L) | Cl) | actual | (95% CI) | actual |
| 1 | 0 | 0X | 3.50-4.06 | 3.50 | 75.7-90.9 | 80.13 |
| 2 (control) | 0 | 1X | 3.68-4.22 | 3.84 | 84.5-101.1 | 100.3 |
| 3 | 0.2 | 1X | 3.30-3.73 | 3.35 | 78.3-90.4 | 85.9 |
| 4 | 0.5 | 1X | 2.67-3.30 | 2.94 | 66.8-83.14 | 76.2 |

0X, glucose concentration was measured at ~1 g/L post Day 6;
1X, glucose concentration was measured at ~3 ± 1 g/L;
2X, glucose concentration was measured at ~6 ± 1 g/L.

This example demonstrated that both glucose concentration and fucose concentration are variables that can be manipulated to modify the levels of TAF and ADCC.

### Example 4

This example demonstrates the impact of fucose on the cell cultures.

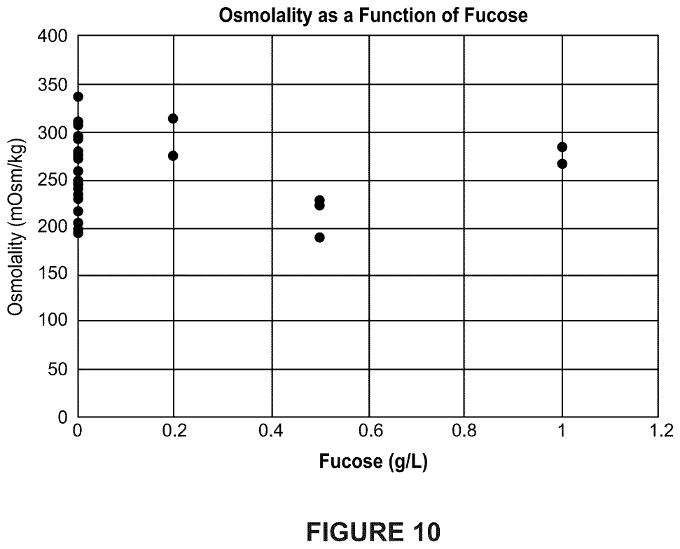

Additional analyses were performed on the cell cultures described above. For example, osmolality of the cell cultures was measured and ranged from about 175 mOsm/kg to about 345 mOsm/kg. A lack of correlation between cell culture osmolality and fucose concentration was observed. See FIG. 10. As shown in this figure, the addition of fucose to the culture medium does not appear to affect the osmolality in any particular way. That the osmolality greatly varied in the control condition (without fucose) suggests that components in the culture medium (other than fucose) actively affect osmolality.

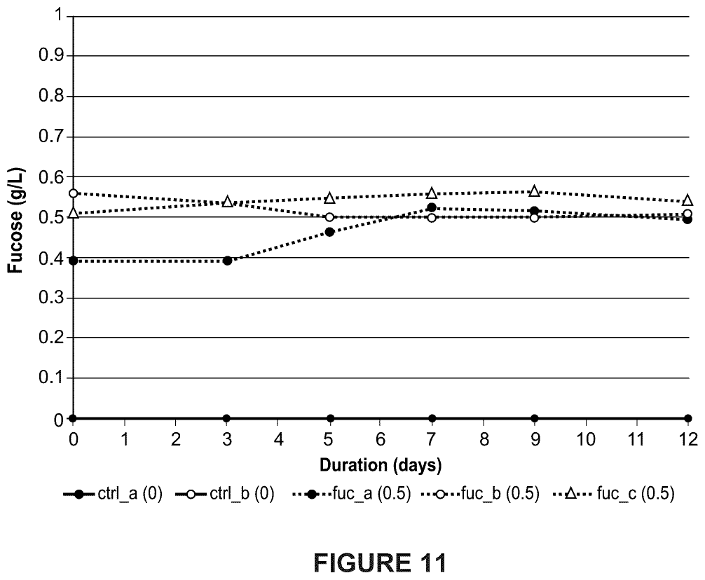

In one experiment of this fucose study, cells were inoculated into one of five bioreactors, two of which contained culture medium without any fucose (ctrl_a and ctrl_b) and three of which contained culture medium with 0.5 g/L fucose (fuc_a, fuc_b, and fuc_c). Media samples from the five cell cultures were collected throughout the 12-day culture period on Day 0, Day 3, Day 5, Day 7, Day 9, and Day 12. The samples were measured for fucose concentration. As shown in FIG. 11, the concentration of fucose did not substantially change throughout the culture period, suggesting the small and/or slow consumption of fucose during this culture process.

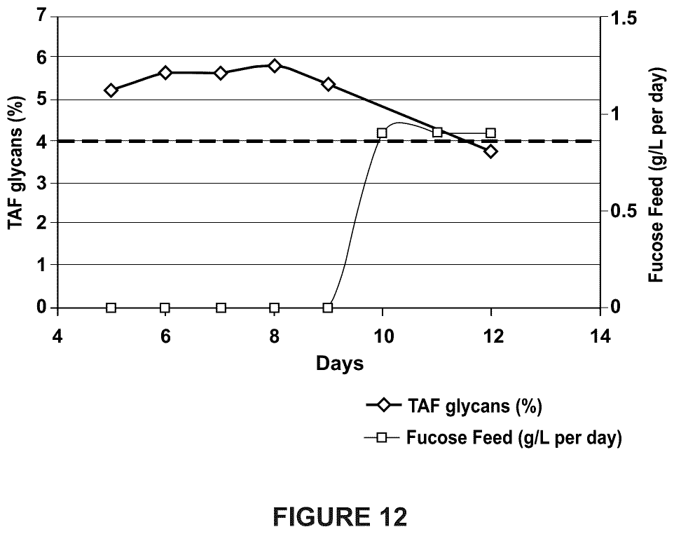

In another experiment of this fucose study, cells were maintained in cell culture for 12 days in a culture medium not containing fucose. In this particular experiment, the pH of the cell culture was disturbed from 7.1 causing the TAF % to increase to about 5.5% from Day 5 to ˜Day 8. TAF % was measured daily starting Day 5. In an attempt to modulate the TAF levels back to the 4.0% target, fucose was added on the 9^{th} day of culture at a feed rate of 0.9 g/L per day. As shown in FIG. 12, the TAF % decreased from about 5.5% upon addition of fucose to the culture medium. The TAF % continued to decrease to ˜3.8% on Day 12. These data suggest that fucose addition may occur late in the culture period and still cause TAF % modulation.

This example demonstrated the impact of fucose concentration in the culture medium on the levels of TAF.

### Example 5

This example demonstrates that maintenance of glucose in the target range can occur late in the culture period.

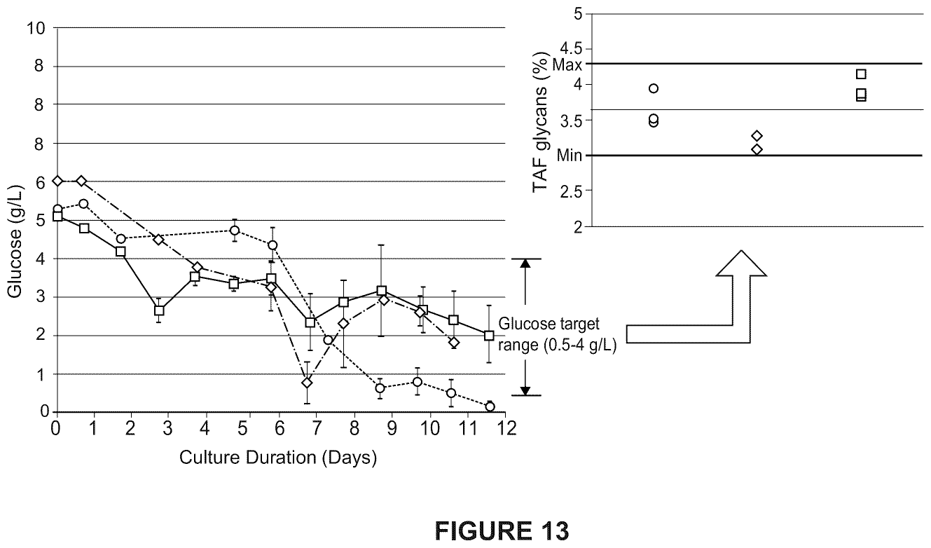

Three experiments were carried out to monitor the timing that the targeted glucose range was reached during a 12-day culture period. For each experiment, the initial glucose concentration of each cell culture ranged from about 5.0 g/L to about 6.0 g/L. Glucose concentrations of the cell culture media were monitored on a daily basis. As shown in FIG. 13, each of the cell cultures reached the targeted glucose concentration (0.5 g/L-4.0 g/L) on different days of the culture period. In one experiment (line with open squares), the targeted range was achieved on Day 2. In a second experiment (dotted line with open diamonds), the targeted range was achieved on Day 4, while in a third experiment (dotted line with open circles), the targeted range was reached on Day 6. Despite these differences, each cell culture achieved the targeted range of TAF % (˜2.0% to ˜4.3%) (see left graph of FIG. 13; third experiment represented by open circles; second experiment represented by open diamonds; first experiment represented by open squares). These data suggest that glucose maintenance can occur later in the culture period to achieve the same TAF levels of cultures maintained earlier during the culture period.

These data demonstrate early and late control of glucose concentrations lead to similar TAF levels.

### Example 6

This example demonstrates the effects of lowering the pH of a cell culture on the level of afucosylation of an antibody with and without the addition of fucose.

A sample of a cell culture was removed from a 2000 L bioreactor and used to inoculate parallel 3 L bioreactors. The 2000 L and 3 L bioreactors were fed using a continuous fed-batch process with Feed A and Feed B for 12 days. Two of the 3 L bioreactors were fed with fucose to a final concentration of 1.0 g/L on Day 5. Afucosylation levels were measured as described in Example 1 and the results are provided in Table 3.

TABLE 3

| pH | Fucose Addition | % afucosylation |
|---|---|---|
| 7.09 | − | 6.29 |
| 7.09 | − | 6.317 |
| 7.07 | − | 6.559 |
| 7.07 | + | 4.384 |
| 7.12 | − | 7.13 |
| 7.13 | − | 7.24 |
| 7.18 | − | 8.365 |
| 7.19 | − | 7.917 |
| 7.18 | + | 6.183 |

As shown in Table 3, in the absence of the addition of fucose, when the pH was below 7.1, the average % afucosylation was 6.39, whereas when the pH was above 7.10 but below 7.15, the average % afucosylation was higher (% afucosylation=7.185). When the pH was above 7.15, the average % afucosylation was yet even higher ((% afucosylation=8.276). Thus, a lower pH was associated with a lower % afucosylation.

When fucose was added to the culture medium (to achieve a final concentration of 1.0 g/L fucose, the % afucosylation was substantially decreased at both a lower pH (7.07) and a higher pH (7.18). For each of these pH levels, when fucose was added, the average decrease in % afucosylation was 2.18%.

These results suggest that lowering the pH of the cell culture medium and/or adding fucose to the cell culture medium leads to a lowered percent afucosylation. When both pH was lowered and fucose was added to the cell culture medium, a greater reduction of percent afucosylation was observed.

### Example 7

This example further demonstrates the effects of lowering the pH of a cell culture on the level of afucosylation of an antibody with and without the addition of fucose.

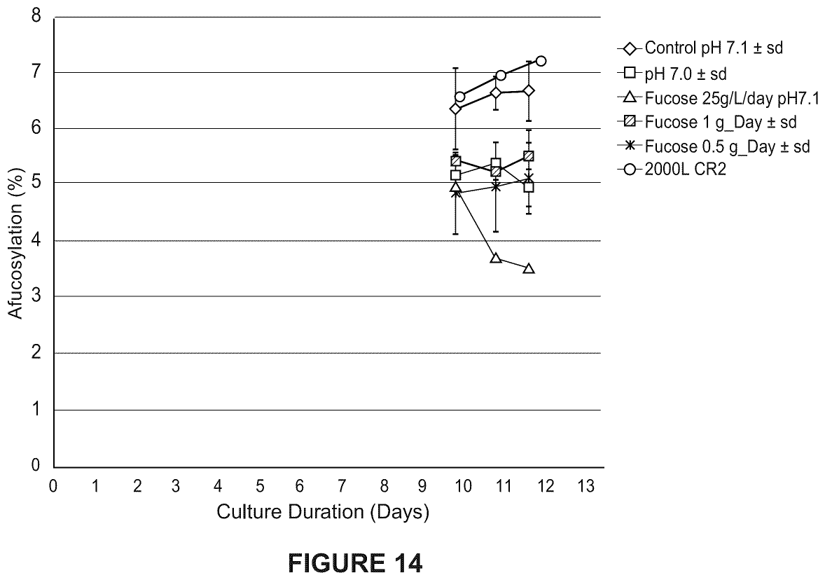

A sample of a cell culture from a 2000 L bioreactor was used to inoculate parallel 3 L bioreactors. The 2000 L and 3 L bioreactors were fed using a continuous fed-batch process using Feed A and Feed B for 12 days. Some of the bioreactors (i.e., cell culture) were fed with fucose to a final concentration of 0.25, 0.5 or 1 g/L on Day 5. Afucosylation levels were measured as described in Example 1 and the results are provided in FIG. 14.

As shown in FIG. 14, the control 3L bioreactor (Control pH 7.1) exhibited a similar level of afucosylation as the original 2000 L bioreactor. Both exhibited % afucosylation of about 6.5% or greater. The addition of fucose at any of the tested levels led to a substantial decrease in % afucosylation (5.5% or lower). The percent afucosylation was lowest with the addition of 0.25 g/L of fucose.

Also, as shown in FIG. 14, lowering the pH from 7.1 to 7.0, without adding fucose to the culture medium, also led to a decrease in afucosylation of at least about 1.0%.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted.

Recitation of ranges of values herein are merely intended to serve as a shorthand method of referring individually to each separate value falling within the range and each endpoint, unless otherwise indicated herein, and each separate value and endpoint is incorporated into the specification as if it were individually recited herein.

All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Preferred embodiments of this disclosure are described herein, including the best mode known to the inventors for carrying out the disclosure. Variations of those preferred embodiments may become apparent to those of ordinary skill in the art upon reading the foregoing description. The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

What is claimed is:

1. A method of producing an antibody composition, wherein the level of total afucosylated (TAF) glycans in the antibody composition is less than about 10%, the method comprising maintaining glycosylation-competent cells in a cell culture medium comprising fucose, wherein fucose is present in the culture medium at a concentration of about 0.17 g/L to about 1.0 g/L.

2. A method of producing an antibody composition, wherein the level of total afucosylated (TAF) glycans in the antibody composition is less than about 10%, the method comprising maintaining glycosylation-competent cells in a cell culture medium comprising fucose, wherein fucose is present in the culture medium at a concentration of about 0.1 g/L to about 1.0 g/L, and wherein the glycosylation-competent cells are not genetically modified to alter activity of an enzyme of the de novo pathway or the salvage pathway.

3. A method of producing an antibody composition, comprising maintaining glycosylation-competent cells in a cell culture medium comprising fucose and glucose, wherein fucose is present in the culture medium at a concentration of about 0.1 g/L to about 1.0 g/L and glucose is added to the cell culture medium according to a glucose feeding schedule that achieves an average glucose concentration of about 10 g/L or less.

4. The method of any one of claims 1 to 3, wherein fucose is present in the culture medium at a concentration less than about 0.75 g/L.

5. The method of claim 4, wherein fucose is present in the culture medium at a concentration less than about 0.6 g/L.

6. The method of claim 5, wherein fucose is present in the culture medium at a concentration of about 0.2 g/L to about 0.5 g/L.

7. The method of any one of claims 1 to 6, wherein the fucose is present in the culture medium the entire duration the glycosylation-competent cells are maintained in cell culture.

8. The method of any one of claims 1 to 6, comprising maintaining the glycosylation-competent cells in a first cell culture medium for an initial time period and subsequently maintaining the glycosylation-competent cells in a second cell culture medium, wherein the first cell culture medium does not comprise fucose at a concentration of about 0.1 g/L to about 1.0 g/L and the second cell culture medium comprises fucose at a concentration of about 0.1 g/L to about 1.0 g/L.

9. The method of claim 8, wherein the initial time period is about 24 to about 72 hours.

10. The method of claim 8, wherein the initial time period is about or greater than about 72 hours but less than or about 156 hours.

11. The method of any one of claims 8 to 10, wherein fucose is added to the first culture medium on the 6^{th} day post-cell culture inoculation to obtain the second cell culture medium.

12. The method of any one of claims 1 to 11, wherein the concentration of fucose fluctuates by about 0.2 g/L or less during the time the glycosylation-competent cells are maintained in the cell culture medium comprising fucose.

13. The method of claim 12, wherein the concentration of fucose fluctuates by about 0.1 g/L or less during the time the glycosylation-competent cells are maintained in the cell culture medium comprising fucose.

14. The method of any one of claims 1 to 13, wherein the cell culture medium comprises an initial glucose concentration for an initial time period.

15. The method of claim 14, wherein the initial glucose concentration is about 1 g/L to about 15 g/L.

16. The method of claim 15, wherein the initial glucose concentration is about 12 g/L±1 g/L.

17. The method of any one of claims 1, 2, and 4 to 16, further comprising adding glucose to the cell culture medium according to a glucose feeding schedule.

18. The method of claim 17, wherein the glucose feeding schedule is initiated at about 4 to about 6 days post-cell culture inoculation.

19. The method of claim 18, wherein the glucose feeding schedule is initiated at about 6 days post-cell culture inoculation.

20. The method of any one of claims 17 to 19, wherein the glucose feeding schedule achieves an average glucose concentration of about 10 g/L or less in the cell culture medium.

21. The method of any one of claims 3 and 17 to 20, wherein the glucose feeding schedule achieves an average glucose concentration of about 9 g/L or less in the cell culture medium.

22. The method of claim 21, wherein the glucose feeding schedule achieves an average glucose concentration of about 6 g/L or less in the cell culture medium.

23. The method of claim 22, wherein the glucose feeding schedule achieves an average glucose concentration of about 0.5 g/L to about 4 g/L in the cell culture medium.

24. The method of any one of claims 17 to 23, wherein the glucose feeding schedule achieves an average glucose concentration based on the concentration of fucose in the cell culture medium.

25. The method of claim 24, wherein the average glucose concentration is calculated based on Formula I:
$$T = 3.354 - 1.388 F + 0.111 G + [ F - 0.43 \; 75 ] \times [ 1.9527 ( F - 0.4375 ) ] \qquad ( Formula 1 )$$
wherein T is the targeted % total afucosylated (TAF) glycans in the antibody composition and is about 2.5% to about 6%, about 2.75% to about 5.5%, or about 3% to about 5%, F is the concentration (g/L) of fucose in the medium, and G is the average glucose concentration (g/L) in the medium.

26. The method of claim 24 or 25, wherein (i) the concentration of fucose is about 0.2±0.1 g/L and the average glucose concentration is about 2 to about 4 g/L; (ii) the concentration of fucose is about 0.5±0.1 g/L and the average glucose concentration is about 3 to about 6 g/L; or (iii) the concentration of fucose is about 0.75±0.1 g/L and the average glucose concentration is about 4.5 to about 9 g/L.

27. The method of any one of claims 1 to 26, wherein the pH of the cell culture medium is about 6.85 to about 7.05.

28. The method of claim 27, wherein the pH of the cell culture medium is about 6.90 to about 7.00.

29. The method of any one of claims 1 and 3 to 34, wherein the glycosylation-competent cells are not genetically modified to alter activity of an enzyme of the de novo pathway or the salvage pathway.

30. The method of claims 2 and 29, wherein the glycosylation-competent cells are not genetically modified to knock-out a gene encoding GDP-keto-6-deoxymannonse-3,5-epimerase, 4-reductase.

31. The method of any one of claims 3 to 30, wherein the level of total afucosylated (TAF) glycans in the antibody composition is less than about 10%.

32. The method of any one of claims 1 to 31, wherein the level of TAF glycans is about 2% to about 6%.

33. The method of claim 32, wherein the level of TAF glycans is about 2% to about 5%.

34. The method of claim 33, wherein the level of TAF glycans in the antibody composition is about 2% to about 4%.

35. The method of any one claims 1 to 34, wherein the level of high mannose glycans in the antibody composition is less than about 3.5%.

36. The method of claim 35, wherein the level of high mannose glycans in the antibody composition is about 0.7% to about 3.0%.

37. The method of any one of claims 1 to 36, wherein the level of afucosylated glycans in the antibody composition is less than about 3.5%.

38. The method of claim 37, wherein the level of afucosylated glycans in the antibody composition is about 0.8% to about 2.8%.

39. The method of any one of claims 1 to 38, wherein the glycosylation-competent cells produce IgG antibodies.

40. The method of claim 39, wherein the glycosylation-competent cells produce IgG1 antibodies.

41. The method of claim 40, wherein the IgG1 antibodies are specific for a tumor-associated antigen.

42. The method of claim 41, wherein the tumor-associated antigen comprises SEQ ID NO. 3.

43. The method of any one of claims 1 to 42, wherein the culture medium does not comprise mannose.

44. An antibody composition produced by the method of any one of claims 1 to 43.

45. A pharmaceutical composition comprising the antibody composition of claim 44 and a pharmaceutically acceptable carrier, diluent or excipient.

46. A cell culture medium comprising:
a. glycosylation-competent cells comprising an exogenous nucleic acid encoding an antibody; and
b. a culture medium comprising fucose at a concentration of about 0.1 g/L to about 1.0 g/L or about 0.17 g/L to about 1.0 g/L.

47. The cell culture medium of claim 46, wherein the glycosylation-competent cells are not genetically modified to alter activity of an enzyme of the de novo pathway or the salvage pathway, optionally, wherein the glycosylation-competent cells are not genetically modified to knock-out a gene encoding GDP-keto-6-deoxymannonse-3,5-epimerase, 4-reductase.

48. The cell culture medium of claim 46 or 47, wherein the culture medium further comprises glucose at a concentration less than about 10 g/L, optionally less than about 9 g/L.

49. The cell culture medium of claim 48, wherein the concentration of glucose is about 6 g/L or less.

50. The cell culture medium of claim 49, wherein the concentration of glucose is about 0.5 g/L to about 4 g/L.

51. The cell culture medium of any one of claims 46 to 50, wherein the pH of the culture medium is about 6.85 to about 7.05.

52. The cell culture medium of claim 51, wherein the pH of the cell culture medium is about 6.90 to about 7.00.

53. The cell culture medium of any one of claims 46 to 52, wherein the cell culture medium does not comprise mannose.

54. The cell culture medium of any one of claims 46 to 53, wherein the antibody is an IgG antibody.

55. The cell culture medium of claim 54, wherein the IgG antibody is an IgG1 antibody.

56. The cell culture medium of claim 55, wherein the IgG1 antibody is specific for a tumor-associated antigen.

57. The cell culture medium of claim 56, wherein the tumor-associated antigen comprises SEQ ID NO: 3.

58. A method of modulating the level of TAF glycans of an antibody composition produced by glycosylation-competent cells, comprising (A) adding fucose to a cell culture medium comprising the glycosylation-competent cells to achieve a fucose concentration of about 0.1 g/L to about 1.0 g/L to decrease the level of TAF glycans; or (B) adding glucose to a cell culture medium comprising the glycosylation-competent cells to achieve a glucose concentration less than about 10 g/L to increase the level of TAF glycans; or (C) both (A) and (B).

59. A method of modulating the level of afucosylated glycans of an antibody composition produced by glycosylation-competent cells, comprising (A) adding fucose to a cell culture medium comprising the glycosylation-competent cells to achieve a fucose concentration of about 0.1 g/L to about 1.0 g/L to decrease the level of afucosylated glycans; or (B) adding glucose to a cell culture medium comprising the glycosylation-competent cells to achieve a glucose concentration less than or about 10 g/L to increase the level of afucosylated glycans; or (C) both (A) and (B).

60. A method of modulating the level of high mannose glycans of an antibody composition produced by glycosylation-competent cells, comprising adding glucose to a cell culture medium comprising the glycosylation-competent cells to achieve a glucose concentration less than or about 10 g/L to increase the level of high mannose glycans.

61. The method of any one of claims 58-60, wherein the glycosylation-competent cells are not genetically modified to alter activity of an enzyme of the de novo pathway or the salvage pathway.

62. The method of claim 61, wherein the glycosylation-competent cells are not genetically modified to knock-out a gene encoding GDP-keto-6-deoxymannonse-3,5-epimerase, 4-reductase.

63. The method of any one of claims 58-62, wherein the level of TAF glycans in the antibody composition is less than or about 10%.

64. The method of claim 63, wherein the level of TAF glycans is about 2% to about 6%.

65. The method of claim 64, wherein the level of TAF glycans is about 2% to about 5%.

66. The method of claim 65, wherein the level of TAF glycans in the antibody composition is about 2% to about 4%.

67. The method of any one of claims 58-66, wherein the level of high mannose glycans in the antibody composition is less than or about 3.5%.

68. The method of claim 67, wherein the level of high mannose glycans in the antibody composition is about 0.7% to about 3.0%.

69. The method of any one of claims 58-68, wherein the level of afucosylated glycans in the antibody composition is less than or about 3.5%.

70. The method of claim 69, wherein the level of afucosylated glycans in the antibody composition is about 0.8% to about 2.8%.

71. The method of any one of claims 58-70, wherein the fucose concentration is about 0.17 g/L to about 1.0 g/L.

72. The method of claim 71, wherein the fucose concentration is about 0.2 g/L to about 0.5 g/L.

73. The method of any one of claims 58 to 72, further comprising adding glucose to the cell culture medium according to a glucose feeding schedule that achieves an average glucose concentration of about 10 g/L, optionally less.

74. The method of claim 73, wherein the average glucose concentration is less than about 6.0 g/L, optionally, less than about 4.0 g/L.

75. The method of claim 73 or 74, wherein the average glucose concentration is based on the fucose concentration of the cell culture medium.

76. The method of claim 75, wherein the average glucose concentration is calculated based on Formula I:
$$T = 3.354 - 1.388 F + 0.111 G + [ F - 0.43 \; 75 ] \times [ 1.9527 ( F - 0.4375 ) ] \qquad Formula 1$$
wherein T is the targeted % total afucosylated (TAF) glycans in an antibody composition and is about 3% to about 5%, F is the concentration (g/L) of fucose in the medium, and G is the average glucose concentration (g/L).

77. A method of modulating the level of afucosylated glycans of an antibody composition produced by glycosylation-competent cells comprising reducing the pH of the cell culture medium by about 0.03 to about 1.2 to reduce the level of afucosylated glycans of an antibody composition by about 0.5% to about 2% or increasing the pH of the cell culture medium by about 0.03 to about 1.2 to increase the level of afucosylated glycans of an antibody composition by about 0.5% to about 2%.

78. The method of claim 77, comprising reducing the pH of the cell culture medium by about 0.05 to about 1.2 to reduce the level of afucosylated glycans of the antibody composition by about 1% to about 2% or increasing the pH of the cell culture medium by about 0.05 to about 1.2 to increase the level of afucosylated glycans of the antibody composition by about 1% to about 2%.

79. The method of claim 77, comprising reducing the pH of the cell culture medium by about 0.03 to about 0.07 to reduce the level of afucosylated glycans of the antibody composition by about 0.5% to about 1.1% or increasing the pH of the cell culture medium by about 0.03 to about 0.07 to increase the level of afucosylated glycans of the antibody composition by about 0.5% to about 1.1%.

80. A method of reducing the level of afucosylated glycans of an antibody composition produced by glycosylation-competent cells by about 1% to about 2%, comprising reducing the pH of the cell culture medium by about 0.05 to about 1.2.

81. The method of claim 80, comprising reducing the pH by about 0.05 to about 0.07 for a reduction in afucosylated glycans of about 1%.

82. The method of claim 80, comprising reducing the pH by about 0.09 to about 1.2 for a reduction in afucosylated glycans of more than about 1.5%.

83. The method of claim 80, comprising culturing the cells at a pH between about 7.10 to about 7.20, optionally about 7.12 to about 7.19.

84. A method of reducing the level of afucosylated glycans of an antibody composition produced by glycosylation-competent cells by about 0.5% to about 1.1%, comprising reducing the pH of the cell culture medium by about 0.03-0.07.

85. The method of claim 84, comprising reducing the pH by about 0.03 to about 0.06 for a reduction in afucosylated glycans of about 0.8%.

86. The method of claim 84, comprising reducing the pH by about 0.05 to about 0.07 for a reduction in afucosylated glycans of about 1%.

87. The method of claim 84, comprising culturing the cells at a pH higher than 7.05 and less than or about 7.15, optionally about 7.07 to about 7.13.

88. A method of increasing the level of afucosylated glycans of an antibody composition produced by glycosylation-competent cells by about 1% to about 2%, comprising increasing the pH of the cell culture medium by about 0.05 to about 1.2.

89. The method of claim 88, comprising increasing the pH by about 0.05 to about 0.07 for a reduction in afucosylated glycans of about 1%.

90. The method of claim 88, comprising increasing the pH by about 0.09 to about 1.2 for a reduction in afucosylated glycans of more than about 1.5%.

91. The method of claim 88, comprising culturing the cells at a pH between about 7.10 to about 7.20, optionally about 7.12 to about 7.19.

92. A method of increasing the level of afucosylated glycans of an antibody composition produced by glycosylation-competent cells by about 0.5% to about 1.1%, comprising increasing the pH of the cell culture medium by about 0.03-0.07.

93. The method of claim 92, comprising increasing the pH by about 0.03 to about 0.06 for a reduction in afucosylated glycans of about 0.8%.

94. The method of claim 92, comprising increasing the pH by about 0.05 to about 0.07 for a reduction in afucosylated glycans of about 1%.

95. The method of claim 92, comprising culturing the cells at a pH higher than 7.05 and lower than or about 7.15, optionally about 7.07 to about 7.13.

96. The method of any one of the preceding claims, wherein the pH of the cell culture medium throughout the culture is greater than 7.0, optionally, higher than 7.05 and lower than 7.2.

97. The method of any one of the preceding claims, wherein the level of afucosylated glycans in the antibody composition is less than about 10%.

98. The method of claim 97, wherein the level of afucosylated glycans in the antibody composition is about 6.2% to about 8.4%.

99. The method of any one of the preceding claims, wherein the temperature changes by less than 2 degrees C. during the culture period.

100. The method of claim 99, wherein the temperature of the culture changes by not more than 1.5 or 1.0 degrees C.

101. The method of any one of the preceding claims, wherein the cell culture medium does not comprise any detectable amounts of manganese or betaine.

102. The method of any one of the preceding claims, wherein the cell culture medium comprises about 0.10 g/L to about 1.0 g/L fucose, optionally, about 0.17 to about 1.0 g/L fucose.

103. The method of claim 102, wherein fucose is present in the culture medium at a concentration less than about 0.75 g/L, less than about 0.6 g/L, or about 0.2 g/L to about 0.5 g/L.

104. The method of any one of the preceding claims, wherein the glycosylation-competent cells are not genetically modified to alter activity of an enzyme of the de novo pathway or the salvage pathway.

105. The method of any one of the preceding claims, wherein glucose is added to the cell culture medium according to a glucose feeding schedule that achieves an average glucose concentration of about 10 g/L or less.

106. A method of modulating the level of TAF glycans of an antibody composition produced by glycosylation-competent cells, comprising modulating the level of afucosylated glycans of the antibody composition in accordance with a method of any one of claims 77-79, reducing the level of afucosylated glycans of the antibody composition in accordance with a method of any one of claims 80-87, or increasing the level of afucosylated glycans of the antibody composition in accordance with a method of any one of claims 88-105.

107. A method of producing an antibody composition, wherein the level of afucosylated glycans in the antibody composition is about 6.2% to about 8.4%, the method comprising maintaining glycosylation-competent cells in a cell culture medium at a pH higher than 7.05 and lower than 7.2,
wherein:
(A) the pH of the cell culture medium changes by less than 0.15 (optionally by less than 0.10) during the culture period or
(B) the temperature of the cell culture medium changes by not more than 2 degrees C. or
(C) the method does not comprise culturing the cells in a cell culture medium comprising manganese or betaine or
(D) a combination of two or three of (A), (B), and (C).

108. The method of claim 107, wherein the pH is maintained at a pH of about 7.07 to about 7.19 during the culture period, optionally, wherein the pH is maintained at about 7.07 or higher and below 7.10, or about 7.10 or higher and below 7.15, or about 7.15 or higher up to about 7.19.


SEQUENCE LISTING (23 sequences)

SEQ ID NO: 1 - protein, 235 aa, synthetic construct
  REGION 1..235: note = Synthetic Polypeptide
MDFQVQIISFLLISASVIMSRGQIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 470 aa, synthetic construct
  REGION 1..470: note = Synthetic Polypeptide
MGWSLILLFLVAVATRVLSQVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 297 aa, synthetic construct
  REGION 1..297: note = Synthetic Polypeptide
MTTPRNSVNGTFPAEPMKGPIAMQSGPKPLFRRMSSLVGPTQSFFMRESKTLGAVQIMNGLFHIALGGLLMIPAGIYAPICVTVWYPLWGGIMYIISGSLLAATEKNSRKCLVKGKMIMNSLSLFAAISGMILSIMDILNIKISHFLKMESLNFIRAHTPYINIYNCEPANPSEKNSPSTQYCYSIQSLFLGILSVMLIFAFFQELVIAGIVENEWKRTCSRPKSNIVLLSAEEKKEQTIEIKEEVVGLTETSSQPKNEEDIEIIPIQEEEEEETETNFPEPPQDQESSPIENDSSP

SEQ ID NO: 4 - protein, 10 aa, synthetic construct
  REGION 1..10: note = Synthetic Polypeptide
  REGION 1..10: note = MISC_FEATURE - LC CDR1
RASSSVSYIH

SEQ ID NO: 5 - protein, 7 aa, synthetic construct
  REGION 1..7: note = Synthetic Polypeptide
  REGION 1..7: note = MISC_FEATURE - LC CDR2
ATSNLAS

SEQ ID NO: 6 - protein, 9 aa, synthetic construct
  REGION 1..9: note = Synthetic Polypeptide
  REGION 1..9: note = MISC_FEATURE - LC CDR3
QQWTSNPPT

SEQ ID NO: 7 - protein, 5 aa, synthetic construct
  REGION 1..5: note = Synthetic Polypeptide
  REGION 1..5: note = MISC_FEATURE - HC CDR1
SYNMH

SEQ ID NO: 8 - protein, 17 aa, synthetic construct
  REGION 1..17: note = Synthetic Polypeptide
  REGION 1..17: note = MISC_FEATURE - HC CDR2
AIYPGNGDTSYNQKFKG

SEQ ID NO: 9 - protein, 12 aa, synthetic construct
  REGION 1..12: note = Synthetic Polypeptide
  REGION 1..12: note = MISC_FEATURE - HC CDR3
STYYGGDWYFNV

SEQ ID NO: 10 - protein, 106 aa, synthetic construct [sequence omitted]

SEQ ID NO: 11 - protein, 121 aa, synthetic construct [sequence omitted]

SEQ ID NO: 12 - protein, 213 aa, synthetic construct
  REGION 1..213: note = Synthetic Polypeptide
  REGION 1..213: note = MISC_FEATURE - VL-IgG Kappa
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 451 aa, synthetic construct
  REGION 1..451: note = Synthetic Polypeptide
  REGION 1..451: note = MISC_FEATURE - VH-IgG1
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 6 aa, synthetic construct
  REGION 1..6: note = Synthetic Polypeptide
  REGION 1..6: note = MISC_FEATURE - LC CDR1
QDVNTA

SEQ ID NO: 15 - skipped

SEQ ID NO: 16 - protein, 9 aa, synthetic construct
  REGION 1..9: note = Synthetic Polypeptide
  REGION 1..9: note = MISC_FEATURE - LC CDR3
QQHYTTPPT

SEQ ID NO: 17 - protein, 8 aa, synthetic construct
  REGION 1..8: note = Synthetic Polypeptide
  REGION 1..8: note = MISC_FEATURE - HC CDR1
GFNIKDTY

SEQ ID NO: 18 - protein, 8 aa, synthetic construct
  REGION 1..8: note = Synthetic Polypeptide
  REGION 1..8: note = MISC_FEATURE - HC CDR2
IYPTNGYT

SEQ ID NO: 19 - protein, 13 aa, synthetic construct
  REGION 1..13: note = Synthetic Polypeptide
  REGION 1..13: note = MISC_FEATURE - HC CDR3
SRWGGDGFYAMDY

SEQ ID NO: 20 - protein, 107 aa, synthetic construct [sequence omitted]

SEQ ID NO: 21 - protein, 120 aa, synthetic construct [sequence omitted]

SEQ ID NO: 22 - protein, 214 aa, synthetic construct
  REGION 1..214: note = Synthetic Polypeptide
  REGION 1..214: note = MISC_FEATURE - VL-IgG Kappa
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 450 aa, synthetic construct
  REGION 1..450: note = Synthetic Polypeptide
  REGION 1..450: note = MISC_FEATURE - VH-IgG1
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG